SEQUENCE LISTING (2 sequences)

SEQ ID NO: 1 - protein, 234 aa, artificial
  CCAR1 polypeptide fragment
MAQFGGQKNPPWATQFTATAVSQPAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQT

SEQ ID NO: 2 - protein, 458 aa, artificial
  CCAR1 polypeptide fragment
VEATYNPNMPFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEISTPTHWSKLDPKTMKVNDLR

(19) United States
(12) Patent Application Publication
(10) Pub. No.: US 2024/0302367 A1
(43) Pub. Date: Sep. 12, 2024

(54) METHODS AND MATERIALS FOR TREATING CANCER

(71) Applicants: The Johns Hopkins University, Baltimore, MD (US); The Board of Trustees of the Leland Stanford Junior University, Stanford, CA (US)

(72) Inventors: Antony Rosen, Pikesville, MD (US); Livia A. Casciola-Rosen, Pikesville, MD (US); David Fiorentino, Stanford, CA (US)

(21) Appl. No.: 18/573,224

(22) PCT Filed: Jun. 7, 2022
(86) PCT No.: PCT/US2022/032462

§ 371 Date: Dec. 21, 2023

Related U.S. Application Data
(60) Provisional application No. 63/214,883, filed on Jun. 25, 2021.
(60) Provisional application No. 63/284,810, filed on Dec. 1, 2021.

(51) Int. Cl. G01N 33/564; A61K 39/00; C07K 16/18; G01N 33/574

(52) U.S. Cl. CPC G01N 33/564; C07K 16/18; G01N 33/57484; A61K 2039/505; G01N 2800/20; G01N 2800/50

(57) ABSTRACT

This document relates to materials and methods for assessing and/or treating mammals (e.g., humans having autoimmune diseases). For example, materials and methods for determining if a mammal (e.g., a human having an autoimmune disease) has one or more antibodies that can be used to identify the mammal as having a lower risk of cancer or as having a higher risk of cancer are provided. Materials and methods for treating a mammal (e.g., a human) identified as having a higher cancer risk for cancer are also provided.

### CROSS-REFERENCE TO RELATED APPLICATIONS

This application claims the benefit of U.S. Patent Application Ser. No. 63/214,883, filed on Jun. 25, 2021, and U.S. Patent Application Ser. No. 63/284,810, filed on Dec. 1, 2021. The disclosure of the prior applications are considered part of (and are incorporated by reference in) the disclosure of this application.

### SEQUENCE LISTING

This document includes a Sequence Listing that has been submitted electronically as an ASCII text file named 44807-0931_ST25.txt. The ASCII text file, created on Jun. 5, 2022, is 7 kilobytes in size. The material in the ASCII text file is hereby incorporated by reference in its entirety.

### TECHNICAL FIELD

This document relates to materials and methods for assessing and/or treating mammals (e.g., humans having autoimmune diseases). For example, this document provides materials and methods for determining if a mammal (e.g., a human having an autoimmune disease) has one or more antibodies that can be used to identify the mammal as having a lower risk of cancer or as having a higher risk of cancer. This document also provides materials and methods for treating a mammal (e.g., a human) identified as having a higher cancer risk for cancer.

### BACKGROUND INFORMATION

A temporal clustering of cancer and dermatomyositis (DM) in a subgroup of DM patients has been appreciated for decades, with diagnosis of cancers particularly prominent in the −3 to +3 year window around DM onset (termed cancer-associated myositis, or CAM; Hill et al., Lancet, 357(9250):96-100 (2000); Sigurgeirsson et al., N. Engl. J. Med., 326(6):363-7 (1992); and Shah et al., Arthritis & Rheumatology, 67(2):317-26 (2015)).

### SUMMARY

This document relates to materials and methods for assessing and/or treating mammals (e.g., humans having autoimmune diseases). For example, this document provides materials and methods for determining if a mammal (e.g., a human having an autoimmune disease) has one or more antibodies described herein (e.g., autoantibodies against a transcription intermediary factor 1-gamma (TIF1-γ) polypeptide and/or one or more decreased cancer risk (DCR) autoantibodies described herein such as, without limitation, an autoantibody against a cell division cycle and apoptosis regulator protein 1 (CCAR1) polypeptide (an anti-CCAR1 autoantibody)) that can be used to identify the mammal as having a lower risk of cancer or as having a higher risk of cancer. This document also provides materials and methods for treating a mammal (e.g., a human) identified as having a higher risk for cancer. For example, this document provides methods and materials for determining that a mammal (e.g., a human having an autoimmune disease) is at a higher risk for cancer and administering one or more cancer treatments (e.g., one or more antibodies such as anti-TIF1-γ antibodies and/or one or more DCR autoantibodies described herein such as, without limitation, anti-CCAR1 antibodies) to treat the mammal.

As demonstrated herein, autoantibody responses are associated with protection of myositis patients from cancer and the cancer risk of the human having myositis can be determined by detecting the presence or absence of one or more autoantibodies. For example, the presence of anti-TIF1-γ autoantibodies (but not one or more DCR autoantibodies described herein such as, without limitation, anti-CCAR1 autoantibodies) in serum obtained from mammals having DM can indicate that the mammal has a higher risk for cancer, while the presence of both anti-TIF1-γ autoantibodies and one or more DCR autoantibodies described herein in serum from mammals having DM can indicate that the mammal has a lower risk for cancer. For example, the presence of both anti-TIF1-γ autoantibodies and one or more of an anti-CCAR1 autoantibody, an autoantibody against a regulator of chromosome condensation 1 (RCC1) polypeptide (an anti-RCC1 autoantibody), an autoantibody against a glutamine amidotransferase like class 1 domain containing 1 (GATD1) polypeptide (an anti-GATD1 autoantibody), an autoantibody against a transducin beta like 1 X-linked receptor 1 (TBL1XR1) polypeptide (an anti-TBL1XR1 autoantibody), an autoantibody against a lysine (K)-specific demethylase 2A (KDM2A) polypeptide (an anti-KDM2A autoantibody), an autoantibody against an inner membrane mitochondrial protein (IMMT) polypeptide (an anti-IMMT autoantibody), an autoantibody against a SRY-box transcription factor 5 (SOX5) polypeptide (an anti-SOX5 autoantibody), an autoantibody against a CDKNIA-interacting zinc finger protein 1 (CIZ1) polypeptide (an anti-CIZ1 autoantibody), an autoantibody against a nuclear valosin-containing protein-like (NVL2) polypeptide (an anti-NVL2 autoantibody), and an autoantibody against a nucleus accumbens-associated protein 1 (NACC1) polypeptide (an anti-NACC1 autoantibody) indicates the mammal having DM is at a decreased risk of having or developing cancer. Having the ability to determine cancer risk in patients (e.g., patients with an autoimmune disease such as, but not limited to, DM) provides a unique and unrealized opportunity to assess cancer risk of a DM patient at the disease onset and throughout the disease course in unique serologic and phenotypic subsets relative to the general population. For example, readily available detection techniques and non-invasive samples can be used to determine cancer risk. The ability to predict increased or decreased cancer risk in patients enables care providers to begin early treatment for patients with increased cancer risk and/or to refrain from subjecting patients with decreased cancer risk to unnecessary workup and/or treatment.

In general, one aspect of this document features methods for determining that a mammal having an autoimmune disease is at a lower risk for cancer. The methods can include, or consist essentially of, detecting the presence or absence of an anti-TIF1-Y antibody in a sample obtained from a mammal having an autoimmune disease; detecting the presence or absence of a DCR antibody in the sample; determining that the mammal has a lower risk for cancer when the presence of the anti-TIF1-γ antibody is detected and when the presence of the DCR antibody is detected. The mammal can be a human. The autoimmune disease can be DM. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, an anti-NACC1 antibody, or any combination thereof. In some cases, the DCR antibody can be an anti-CCAR1 antibody. The lower risk for cancer can include a lower risk for having cancer. The lower risk for cancer can include a lower risk for developing cancer. The cancer can be prostate cancer, breast cancer, leukemia cancer, lung cancer, melanoma, uterine cancer, kidney cancer, thyroid cancer, ovarian cancer, or colon cancer.

In another aspect, this document features methods for determining that a mammal having an autoimmune disease is at a higher risk for cancer. The methods can include, or consist essentially of, detecting the presence or absence of an anti-TIF1-γ antibody in a sample obtained from a mammal having an autoimmune disease; detecting the presence or absence of a DCR antibody in the sample; determining that the mammal has a higher risk for cancer when the presence of the anti-TIF1-γ antibody is detected and when the absence of the DCR antibody is detected. The mammal can be a human. The autoimmune disease can be DM. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, and an anti-NACC1 antibody. The lower risk for cancer can include a lower risk for having cancer. The lower risk for cancer can include a lower risk for developing cancer. The cancer can be prostate cancer, breast cancer, leukemia cancer, lung cancer, melanoma, uterine cancer, kidney cancer, thyroid cancer, ovarian cancer, or colon cancer.

In another aspect, this document features methods for treating a mammal having cancer. The methods can include, or consist essentially of, detecting a presence of an anti-TIF1-γ antibody and an absence of a DCR antibody in a sample obtained from a mammal having cancer; and administering an inhibitor of a CCAR1 polypeptide to the mammal. The mammal can be a human. The mammal can have an autoimmune disease. The autoimmune disease can be DM. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, an anti-NACC1 antibody, or any combination thereof. In some cases, the DCR antibody can be an anti-CCAR1 antibody. The inhibitor of the CCAR1 polypeptide is an anti-CCAR1 antibody. The cancer can be prostate cancer, breast cancer, leukemia cancer, lung cancer, melanoma, uterine cancer, kidney cancer, thyroid cancer, ovarian cancer, or colon cancer.

In another aspect, this document features methods for treating a mammal having cancer. The methods can include, or consist essentially of, administering an inhibitor of a CCAR1 polypeptide to a mammal identified as having a presence of an anti-TIF1-γ antibody and an absence of a DCR antibody. The mammal can be a human. The mammal can have an autoimmune disease. The autoimmune disease can be DM. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, an anti-NACC1 antibody, or any combination thereof. In some cases, the DCR antibody can be an anti-CCAR1 antibody. The inhibitor of the CCAR1 polypeptide is an anti-CCAR1 antibody. The cancer can be prostate cancer, breast cancer, leukemia cancer, lung cancer, melanoma, uterine cancer, kidney cancer, thyroid cancer, ovarian cancer, or colon cancer.

In another aspect, this document features methods for selecting a mammal for increased monitoring. The methods can include, or consist essentially of, detecting the presence or absence of an anti-TIF1-γ antibody in a sample obtained from a mammal; detecting the presence or absence of a DCR antibody in the sample; selecting the mammal for increased monitoring when the presence of said anti-TIF1-γ antibody is detected and when the absence of the DCR antibody is detected. The mammal can be a human. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, an anti-NACC1 antibody, or any combination thereof. In some cases, the DCR antibody can be an anti-CCAR1 antibody.

In another aspect, this document features methods for selecting a mammal for further diagnostic testing. The methods can include, or consist essentially of, detecting the presence or absence of an anti-TIF1-γ antibody in a sample obtained from a mammal; detecting the presence or absence of a DCR antibody in the sample; selecting the mammal for further diagnostic testing when the presence of the anti-TIF1-γ antibody is detected and when the absence of the DCR antibody is detected. The mammal can be a human. The DCR antibody can be an anti-CCAR1 antibody, an anti-GATD1 antibody, an anti-TBL1XR1 antibody, an anti-KDM2A antibody, an anti-IMMT antibody, an anti-SOX5 antibody, an anti-C1Z1 antibody, an anti-NVL2 antibody, an anti-NACC1 antibody, or any combination thereof. In some cases, the DCR antibody can be an anti-CCAR1 antibody.

In another aspect, this document features methods for detecting a presence or absence of an anti-CCAR1 antibody in a sample. The methods can include, or consist essentially of, contacting a sample to a CCAR1 polypeptide fragment consisting essentially of or consisting of the amino acid sequence set forth in SEQ ID NO: 1 or SEQ ID NO:2 under conditions that allow a complex to form between the CCAR1 polypeptide fragment and the anti-CCAR1 antibody; and detecting the complex where the complex is indicative of the presence of the anti-CCAR1 antibody. The CCAR1 polypeptide fragment can be immobilized on an enzyme-linked immunosorbent assay (ELISA) plate.

In another aspect, this document features ELISA plates where a CCAR1 polypeptide fragment consisting essentially of or consisting of the amino acid sequence set forth in SEQ ID NO: 1 or SEQ ID NO:2 can be immobilized on the ELISA plate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B are blots of ELISAs and immunoprecipitation (IP) assays to detect TIF1-γ and CCAR1 antibodies. FIG. 1A is a comparison of ELISA and IP/blot assays to detect anti-TIF1-γ antibodies. IPs were performed using TIF1-γ-transfected lysates and plasma from 11 anti-TIF1-γ positive dermatomyositis patients (lanes 3-13) or from 2 healthy controls (lanes 1 & 2). The presence of anti-TIF1-γ antibodies was detected by blotting with an anti-TIF1-γ monoclonal antibody. The same samples were also tested using a commercially available ELISA assay to determine the presence of anti-TIF1-γ antibodies. Results of this assay (units) are noted above the IP panel. FIG. 1B is an IP assay to identify patients with anti-cell division cycle and apoptosis regulator protein (CCAR1) antibodies. Plasma samples from 7 anti-TIF1-γ-positive dermatomyositis patients (lanes 15-21) and 6 healthy controls (lanes 22-27) were used to immunoprecipitate 35S-methionine-labeled CCAR1 generated by in vitro transcription/translation. Dermatomyositis samples 15-19 were anti-CCAR1 positive, 20 and 21 were anti-CCAR1 negative, as were the controls. An anti-FLAG calibrator IP, performed using an anti-FLAG monoclonal antibody (Sigma), is shown in lane 14.

FIGS. 2A-2B show IPs performed using radiolabeled cell lysates from patients who had not had cancer (No cancer-FIG. 2A) or who had cancer within 3 years (Cancer within 3 years-FIG. 2B).

FIGS. 3A-3D show graphs and tables related to an increasing number of antibody targets observed (FIGS. 3A-3C) with lengthening time between DM diagnosis and cancer emergence (FIG. 3D). FIG. 3A shows mean computational traces of IPs performed using samples from anti-TIF1-γ-positive DM patients with (n=18) or without (n=18) cancer. FIG. 3B shows IPs arranged according to cancer timing. FIG. 3C is a plot that shows the number of autoantibody targets as a function of cancer status. FIG. 3D is a plot showing the timing (in years) of individual cancers diagnosed after DM-symptom onset stratified by anti-CCAR1 antibody status.

FIGS. 4A-4E show novel autoantibody discovery in anti-TIF1-γ-positive DM patients without cancer. FIG. 4A is an IP blot of patient samples with and without cancer. FIG. 4B shows autoantibody targets identified by mass spectrometry in 5 different anti-TIF1-γ-positive patient samples all without a detected cancer. FIG. 4C shows the frequency of antibody specificities identified in FIG. 4B across anti-TIF1-γ-positive DM patient cohorts compared to healthy controls. FIG. 4D shows an immunoblot of HeLa and A431 cell lysates performed with commercial antibodies against TIF1-γ and CCAR1. HC, healthy control. FIG. 4E shows interaction between CCAR1 and TIF1-γ. Co-IPs were performed using antibodies against CCAR1 (upper panel, 2 left lanes) or TIF1-γ (lower panel, 2 left lanes). Detection of the IPs was performed by immunoblotting with anti-TIF1-γ (upper panel, 2 left lanes) or anti-CCAR1 (lower panel, 2 left lanes) antibodies. Control IPs, performed using Protein A beads only, were performed and immunoblotted as above. IPs were performed in duplicate. These data are representative of that obtained in 2 additional experiments.

FIGS. 5A-5D show that protection against cancer was associated with combinatorial expression of autoantibodies. FIGS. 5A-5B shows the number, identity, and frequency of unique autoantibody combinations in patients with (FIG. 5A) or without (FIG. 5B) cancer within 3 years of DM onset. The vertical histogram above the matrix shows the frequency of specific autoantibodies in the cohort of anti-TIF1-γ-positive patients, in order of decreasing magnitude. The y-axis of those plots denotes the number of patients. In the matrix itself, each row represents one autoantibody combination. Grey circles denote absence of a specific antibody, black circles denote presence, and when multiple specificities were present in a combination, black lines connect the circles. The frequency of each combination is shown in the horizontal bar plots; the x-axis denotes the number of patients. FIG. 5C shows the mean number of autoantibody specificities in anti-TIF1-γ-positive DM patients in whom cancer does or does not emerge. Data at 1 year, 3 years, 5 years, and ever after DM diagnosis are shown (mean+/−s.e.m., obtained by a bootstrapping procedure (n=10000 samples)). P-values were obtained by 2-tailed independent 2-sample t-test; *=p<0.05, **=p<0.01, ***=p<0.001, ****=p<1e-3. FIG. 5D shows the proportion of DM patients in whom cancer does (“cancer”) or does not (“no cancer”) emerge within 3 years of DM diagnosis that have anti-TIF1-γ antibodies only (number of specificities=0) or anti-TIF1-γ plus additional specificities (number of specificities=1-5). Histograms of antibody count in excess of TIF1-γ were computed and are shown. The cumulative distribution of antibody count in excess of TIF1-γ was also computed for cancer vs. no-cancer groups, and superimposed on the histograms (thin traces at top).

FIGS. 6A-6D show that protection against cancer was associated with combinatorial expression of autoantibodies. Number, identity, and frequency of unique autoantibody combinations in patients with (FIG. 6A) or without cancer (FIG. 6B) within 1 year of DM onset, or with (FIG. 6C) or without (FIG. 6D) cancer within 5 years of DM onset. The vertical histogram above the matrix shows the frequency of specific autoantibodies in the cohort of anti-TIF1-γ-positive patients, in order of decreasing magnitude. The y-axis of those plots denotes number of patients. In the matrix itself, each row represents one autoantibody combination. Grey circles denote absence of a specific antibody, black circles denote presence, and when multiple specificities are present in a combination, black lines connect the circles. The frequency of each combination is shown in the horizontal bar plots; the x-axis denotes the number of patients.

FIG. 7 depicts a model of an exemplary relationship between cancer fitness and immune response.

FIG. 8 is a plot of ELISA results from CCAR1 antibody detection validation.

### DETAILED DESCRIPTION

Patients with autoimmune diseases (e.g., DM and other autoimmune diseases) have distinctive autoantibodies that are associated with unique clinical phenotypes. For example, the presence or absence of autoantibodies in DM patients can predict increased or decreased cancer risk as compared to the general population.

This document provides materials and methods for assessing the cancer risk of mammal (e.g., humans) having autoimmune diseases (e.g., DM) as compared to the general population (e.g., healthy patients and/or patients that do not have an autoimmune disease). The cancer risk (e.g., a lower cancer risk or a higher cancer risk) of a mammal having an autoimmune disease can be determined based, at least in part, on the presence or absence of one or more antibodies in a sample obtained from the mammal. In some cases, the materials and methods described herein can be used to determine that a mammal (e.g., a mammal having an autoimmune disease such as DM) has a higher cancer risk (e.g., a higher risk for having cancer or a higher risk for developing cancer). As described herein, the presence of anti-TIF1-γ autoantibodies (but not one or more DCR autoantibodies described herein such as, without limitation, anti-CCAR1 autoantibodies) in serum obtained from mammals having DM can indicate that the mammal has a higher risk for cancer. Also as described herein, the presence of both anti-TIF1-γ autoantibodies and one or more DCR autoantibodies described herein in serum from mammals having DM can indicate that the mammal has a lower risk for cancer. As used herein, the term “DCR autoantibody” refers to any one or more of anti-CCAR1 autoantibodies, anti-RCC1 autoantibodies, anti-GATD1 autoantibodies, anti-TBL1XR1 autoantibodies, anti-KDM2A autoantibodies, anti-IMMT autoantibodies, anti-SOX5 autoantibodies, anti-CIZ1 autoantibodies, anti-NVL2 autoantibodies, and anti-NACC1 autoantibodies. An antibody that is associated with one or more autoimmune diseases (e.g., DM) can be any appropriate antibody. In some cases, the antibody can be an autoantibody. In some cases, antibodies can be DM-specific autoantibodies. In some cases, antibodies can be cross-reactive antibodies. Examples of antibodies that are associated with the risk of cancer in mammals having one or more autoimmune diseases include, without limitation, anti-TIF1-γ antibodies, anti-CCAR1 antibodies, anti-RCC1 antibodies, anti-GATD1 antibodies, anti-TBL1XR1 antibodies, anti-KDM2A antibodies, anti-IMMT antibodies, anti-SOX5 antibodies, anti-C1Z1 antibodies, anti-NVL2 antibodies, and anti-NACC1 antibodies. Exemplary anti-TIF1-γ antibodies, anti-CCAR1 antibodies, anti-RCC1 antibodies, anti-GATD1 antibodies, anti-TBL1XR1 antibodies, anti-KDM2A antibodies, anti-IMMT antibodies, anti-SOX5 antibodies, anti-CIZ1 antibodies, anti-NVL2 antibodies, and anti-NACC1 antibodies are described, for example, in Example 1.

Any appropriate mammal can be assessed and/or treated as described herein. In some cases, a mammal that can be assessed and/or treated as described herein can have one or more autoimmune diseases (e.g., DM). Examples of mammals that can be assessed and/or treated as described herein include, without limitation, humans, non-human primates such as monkeys, dogs, cats, horses, cows, pigs, sheep, mice, and rats. In some cases, a human can be assessed and/or treated as described herein. For example, the presence or absence of one or more antibodies described herein (e.g., anti-TIF1-γ antibodies and one or more DCR antibodies described herein such as, without limitation, anti-CCAR1 antibodies) in a sample obtained from a human having DM can be used to determine the cancer risk of the human. In some cases, humans identified as having a higher risk of cancer as described herein and/or identified as having cancer can be treated. For example, one or more antibodies described herein (e.g., anti-TIF1-γ antibodies and/or one or more DCR antibodies described herein such as, without limitation, anti-CCAR1 antibodies) can be administered to a mammal identified as having a higher risk for cancer and/or identified as having a cancer to treat the mammal.

When assessing and/or treating a mammal (e.g., a human) having an autoimmune disease as described herein, the autoimmune disease can be any autoimmune disease. In some cases, an autoimmune disease can be a cancer-associated rheumatic syndrome. An example of an autoimmune disease that can assessed as described herein includes, without limitation, DM. In some cases, materials and methods described herein can be used to assess and/or treat a mammal having DM.

In some cases, the methods described herein can include identifying a mammal (e.g., a human) as having an autoimmune disease. Any appropriate method can be used to identify a mammal having an autoimmune disease. Examples of methods that can be used to identify mammals (e.g., humans) having an autoimmune disease include, without limitation, autoantibody (e.g., antinuclear) tests, physical diagnoses, genetic screening, muscle imaging, muscle biopsies, skin biopsies, strength testing, and detection of blood parameters like muscle enzymes and inflammatory markers.

Any appropriate sample from a mammal (e.g., a human) can be used to detect the presence or absence of one or more antibodies described herein (e.g., anti-TIF1-γ antibodies and one or more DCR antibodies described herein such as, without limitation, anti-CCAR1 antibodies) in the mammal (e.g., a human). For example, biological samples such as fluid samples (e.g., blood, serum, or plasma) can be obtained from a human, and the presence or absence of anti-TIF1-γ antibodies and/or one or more DCR antibodies described herein (e.g., anti-CCAR1 antibodies) can be detected. In some cases, a blood sample can be obtained from a mammal and used to detect the presence or absence of anti-TIF1-γ antibodies and/or one or more DCR antibodies described herein (e.g., anti-CCAR1 antibodies). In some cases, a serum sample can be obtained from a mammal and used to detect the presence or absence of anti-TIF1-γ antibodies and/or one or more DCR antibodies described herein (e.g., anti-CCAR1 antibodies). In some cases, a plasma sample can be obtained from a mammal and used to detect the presence or absence of anti-TIF1-γ antibodies and/or one or more DCR antibodies described herein (e.g., anti-CCAR1 antibodies).

Any appropriate method can be used to detect the presence or absence of one or more antibodies described herein (e.g., anti-TIF1-γ antibodies and one or more DCR antibodies described herein such as, without limitation, anti-CCAR1 antibodies). Methods of detecting the presence or absence of an antibody include, without limitation, immunoprecipitation (IP), western blotting, ELISA assays, Elispot assays, chemilumionesence assays, and line immunoprecipitation assays.

In some cases, the presence or absence of an antibody described herein (e.g., an anti-CCAR1 antibody) can be detected using an ELISA assay. For example, ELISA can be performed using one or more fragments of a polypeptide that is recognized by a DCR antibody (e.g., one or more CCAR1 polypeptide fragments). For example, a CCAR1 polypeptide fragment can be used in an ELISA assay to detect an anti-CCAR1 antibody. In some cases, a polypeptide fragment can be a purified polypeptide fragment. In some cases, a polypeptide fragment can be a recombinant polypeptide fragment. In some cases, a polypeptide fragment can be non-naturally occurring polypeptide fragment. In some cases, a polypeptide fragment can be an N-terminal fragment of a polypeptide. In some cases, a CCAR1 polypeptide fragment that can be used in an ELISA assay to detect the presence or absence of an anti-CCAR1 antibody can comprise, consist essentially of, or consist of the amino acid sequences set forth below:

TABLE 1
Exemplary polypeptide fragments.

| polypeptide | | | |
|---|---|---|---|
| recognized | | | |
| by a DCR | | | SEQ |
| antibody | aa | Sequence | ID NO |
| CCAR1 | 1-234 | MAQFGGQKNPPWATQFTATAVSQPAALGVQ | 1 |
| | | QPSLLGASPTIYTQQTALAAAGLTTQTPAN | |
| | | YQLTQTAALQQQAAAAAAALQQQYSQPQQA | |
| | | LYSVQQQLQQPQQTLLTQPAVALPTSLSLS | |
| | | TPQPTAQITVSYPTPRSSQQQTQPQKQRVF | |
| | | TGVVTKLHDTFGFVDEDVFFQLSAVKGKTP | |
| | | QVGDRVLVEATYNPNMPFKWNAQRIQTLPN | |
| | | QNQSQTQPLLKTPPAVLQPIAPQT | |
| CCAR1 | 188-645 | VEATYNPNMPFKWNAQRIQTLPNQNQSQTQ | 2 |
| | | PLLKTPPAVLQPIAPQTTFGVQTQPQPQSL | |
| | | LQAQISAASITPLLQTQPQPLLQQPQQKAG | |
| | | LLQPPVRIVSQPQPARRLDPPSRFSGRNDR | |
| | | GDQVPNRKDDRSRERERERRRSRERSPQRK | |
| | | RSRERSPRRERERSPRRVRRVVPRYTVQFS | |
| | | KFSLDCPSCDMMELRRRYQNLYIPSDFFDA | |
| | | QFTWVDAFPLSRPFQLGNYCNFYVMHREVE | |
| | | SLEKNMAILDPPDADHLYSAKVMLMASPSM | |
| | | EDLYHKSCALAEDPQELRDGFQHPARLVKF | |
| | | LVGMKGKDEAMAIGGHWSPSLDGPDPEKDP | |
| | | SVLIKTAIRCCKALTGIDLSVCTQWYRFAE | |
| | | IRYHRPEETHKGRTVPAHVETVVLFFPDVW | |
| | | HCLPTRSEWETLSRGYKQQLVEKLQGERKE | |
| | | ADGEQDEEEKDDGEAKEISTPTHWSKLDPK | |
| | | TMKVNDLR | |

In some cases, a polypeptide fragment that can be used in an ELISA assay to detect the presence or absence of a DCR autoantibody described herein such as, without limitation, an anti-CCAR1 antibody can have a sequence that is not 100% identical to the sequences set forth in Table 1, but retains the ability to bind to the antibody. For example, a polypeptide fragment that includes one or more (e.g., one, two, three, four, five, or more) amino acid substitutions relative to a polypeptide fragment set forth in Table 1 can be used in an ELISA assay to detect the presence or absence of a DCR autoantibody described herein such as, without limitation, an anti-CCAR1 antibody. An amino acid substitution can be made, in some cases, by selecting a substitution that does not differ significantly in its effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be made within a polypeptide fragment that can be used in an ELISA assay to detect the presence or absence of a DCR autoantibody described herein such as, without limitation, an anti-CCAR1 antibody include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, a CCAR1 polypeptide fragment that can be used in an ELISA assay to detect the presence or absence of an anti-CCAR1 antibody that consists essentially of an amino acid sequence set forth in SEQ ID NO:1 or SEQ ID NO:2 is a CCAR1 polypeptide fragment that has zero, one, or two amino acid substitutions within the articulated sequence of the sequence identifier (e.g., SEQ ID NO:1 or SEQ ID NO:2), has zero, one, two, three, four, or five amino acid residues preceding the articulated sequence of the sequence identifier (e.g., SEQ ID NO:1 or SEQ ID NO:2), and/or has zero, one, two, three, four, or five amino acid residues following the articulated sequence of the sequence identifier (e.g., SEQ ID NO:1 or SEQ ID NO:2), provided that the CCAR1 polypeptide fragment has the ability to target (e.g., target and bind to) an anti-CCAR1 antibody.

In some cases, a CCAR1 polypeptide fragment can be used in an ELISA assay to detect the presence or absence of an anti-CCAR1 antibody. For example, a sample (e.g., a sample obtained from a mammal such as a human having an autoimmune disease such as DM) can be contacted to an ELISA plate having one or more (e.g., one, two, three, four, or more) CCAR1 polypeptide fragments described herein (e.g., one or more CCAR1 polypeptide fragments consisting essentially of or consisting of SEQ ID NO: 1 or SEQ ID NO:2) immobilized on the ELISA plate. The contacting step can be performed under conditions where, when an anti-CCAR1 antibody is present, the anti-CCAR1 antibody can form a complex with an immobilized CCAR1 polypeptide fragment. The method also can include detecting the presence or absence of complexes formed from an immobilized CCAR1 polypeptide fragment and an anti-CCAR1 antibody where the presence of a complex is indicative of the presence of the anti-CCAR1 antibody in the sample and the absence of a complex is indicative the of the absence of the anti-CCAR1 antibody in the sample. In some cases, an ELISA assay to detect the presence or absence of an anti-CCAR1 antibody can be as described in Example 2.

Also provided herein are ELISA plates comprising one or more polypeptide fragments described herein (e.g., one or more CCAR1 polypeptide fragments consisting essentially of or consisting of SEQ ID NO:1 or SEQ ID NO:2). For example, one or more polypeptide fragments described herein (e.g., one or more CCAR1 polypeptide fragments consisting essentially of or consisting of SEQ ID NO:1 or SEQ ID NO:2) can be immobilized on an ELISA plate (e.g., the bottom of one or more wells of an ELISA plate).

In some cases, the presence of an antibody described herein (e.g., an anti-TIF1-γ antibody or one or more DCR antibodies described herein such as, without limitation, an anti-CCAR1 antibody) can be any detectable level of the antibody. In some cases, the presence of an antibody can be any detectable level that is higher than a reference level of an antibody. In some cases, the presence of an antibody can be a detectable level that is at least 1, 2, 3, 4, 5, or more standard deviations above a reference level of the antibody.

In some cases, the absence of an antibody described herein (e.g., an anti-TIF1-γ antibody or one or more DCR antibodies described herein such as, without limitation, an anti-CCAR1 antibody) can be any non-detectable level of the antibody. In some cases, the absence of an antibody can be any level that is about the same or lower than a reference level of an antibody.

In some cases, a reference level of an antibody described herein (e.g., an anti-TIF1-γ antibody or one or more DCR antibodies described herein such as, without limitation, an anti-CCAR1 antibody) can be the level of the antibody present in a reference mammal (e.g., a human) that does not exhibit the autoimmune disorder. In some cases, a reference level of an antibody can be the level of the antibody present in the mammal prior to onset of the autoimmune disorder.

Once the presence or absence of one or more antibodies described herein (e.g., anti-TIF1-γ antibodies and/or one or more DCR antibodies described herein such as, without limitation, anti-CCAR1 antibodies) has been detected in a sample (e.g., a biological sample) obtained from a mammal (e.g., a human), the mammal can be assessed to determine the cancer risk of the mammal. When a mammal is identified as being at higher risk for cancer as described herein (e.g., based, at least in part, on the presence or absence of anti-TIF1-Y antibodies and/or one or more DCR antibodies described herein such as, without limitation, anti-CCAR1 antibodies), a treatment option for the mammal can be selected, and optionally, the mammal can be treated using the selected treatment option.

In some cases, the presence or absence of one or more anti-TIF1-γ antibodies in a sample obtained from a mammal (e.g., a human) having an autoimmune disease (e.g., DM) can be used to determine the cancer risk of the mammal. For example, the presence of an anti-TIF1-γ antibody in a biological sample obtained from a mammal having an autoimmune disease (e.g., DM) can indicate that the mammal has a higher risk for cancer (e.g., as compared to the general population or as compared to a mammal lacking the presence of an anti-TIF1-γ antibodies). For example, the absence of an anti-TIF1-γ antibody in a biological sample obtained from a mammal having an autoimmune disease (e.g., DM) can indicate that the mammal has a lower risk for cancer.

In some cases, the presence or absence of one or more anti-TIF1-γ antibodies and one or more DCR antibodies described herein such as, without limitation, anti-CCAR1 antibodies in a sample obtained from a mammal (e.g., a human) having an autoimmune disease (e.g., DM) can be used to determine the cancer risk of the mammal. For example, the presence of an anti-TIF1-γ antibody and the presence of one or more DCR antibodies described herein (e.g., an anti-CCAR1 antibody) in a biological sample obtained from a mammal having an autoimmune disease (e.g., DM) can indicate that the mammal has a lower risk for cancer (e.g., as compared to the general population, as compared to a mammal lacking the presence of an anti-TIF1-γ antibodies and lacking the presence of one or more DCR antibodies described herein such as, without limitation, anti-CCAR1 antibodies, or as compared to a mammal having the presence of an anti-TIF1-γ antibodies and lacking the presence of one or more DCR antibodies described herein such as, without limitation, anti-CCAR1 antibodies). For example, the presence of an anti-TIF1-γ antibody and the absence of a DCR autoantibody described herein (e.g., the absence of any of the DCR antibodies described herein) in a biological sample obtained from a mammal having an autoimmune disease (e.g., DM) can indicate that the mammal has a higher risk for cancer.

When a mammal (e.g., a human) having an autoimmune disease (e.g., DM) is identified as having lower risk for cancer as described herein (e.g., based, at least in part, on the presence of anti-TIF1-γ antibodies and one or more DCR antibodies described herein such as, without limitation, anti-CCAR1 antibodies in a sample obtained from a mammal) or as having higher risk for cancer as described herein (e.g., based, at least in part, on the presence of anti-TIF1-γ antibodies and the absence of any of the DCR antibodies described herein in a sample obtained from a mammal), the mammal can be selected for monitoring and/or for further diagnostic testing. Examples of increased monitoring can include, without limitation, undergoing more frequent cancer screening including, but not limited to, imaging techniques such as mammograms or CTs, physical examination, laboratory tests, positron emission tomography (PET) scanning, colonoscopy, and/or detection of one or more tumor markers (e.g., PSA). In some cases, a mammal can be selected for increased monitoring for a particular cancer. For example, increased monitoring for breast cancer can include more frequent and/or alternating MRI and mammograms. For example, increased monitoring for lung cancer can include obtaining and investigating more frequent chest CT images. For example, increased monitoring for prostate cancer can include more frequent PSA tests. In some cases, when the presence of an anti-TIF1-γ antibody and the presence of one or more DCR antibodies described herein (e.g., an anti-CCAR1 antibody) are detected in a sample obtained from a human having an autoimmune disease (e.g. DM), the human can be selected for monitoring and/or for further diagnostic testing. In some cases, when the presence of an anti-TIF1-γ antibody and the absence of any of the DCR antibodies described herein are detected in a sample obtained from a human having an autoimmune disease (e.g. DM), the human can be selected for monitoring and/or for further diagnostic testing.

When a mammal (e.g., a human) having an autoimmune disease (e.g., DM) is identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal), the mammal can be selected for therapeutic intervention. In some cases, a therapeutic intervention can include anti-TIF1-γ antibodies and/or one or more DCR antibodies described herein such as, without limitation, anti-CCAR1 antibodies. In some cases, a therapeutic intervention can include radiation therapy. In some cases, a therapeutic intervention can include surgery. In some cases, a therapeutic intervention can include one or more anti-cancer agents. Examples of anti-cancer agents include, without limitation, adoptive T cell therapy (e.g., chimeric antigen receptors and/or T cells having wild-type or modified T cell receptors), chemotherapeutic agents, immune checkpoint inhibitors, targeted therapies (e.g., agents that can target a particular genetic lesion, such as a translocation or mutation), and signal transduction inhibitors. In some cases, when the presence of an anti-TIF1-γ antibody and the absence of a DCR autoantibody described herein (e.g., the absence of any of the DCR antibodies described herein) are detected in a sample obtained from a human having an autoimmune disease (e.g. DM), the human can be selected for therapeutic intervention.

This document also provides materials and methods for treating mammals (e.g., humans having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer. For example, one or more inhibitors of a CCAR1 polypeptide (e.g., anti-CCAR1 antibodies) can be administered to a mammal identified as having a higher risk for cancer as described herein and/or identified as having a cancer to treat the mammal. For example, one or more fragments (e.g., immunogenic fragments) of a CCAR1 polypeptide can be administered to a mammal identified as having a higher risk for cancer as described herein and/or identified as having a cancer to induce production of anti-CCAR1 antibodies within the mammal to treat the mammal. In some cases, treating a mammal identified as having a higher risk for cancer can be effective to slow or prevent development of a cancer. In some cases, treating a mammal identified as having cancer can be effective to reduce or eliminate the number of cancer cells within the mammal. In some cases, materials and methods described herein also can include identifying the mammal as having a higher risk for cancer and/or identifying the mammal as having cancer.

In some cases, the methods and materials provided herein can be used to reduce or eliminate the number of cancer cells present within a mammal (e.g., a human) having cancer. For example, a mammal in need thereof (e.g., a mammal having cancer) can be administered one or more inhibitors of a CCAR1 polypeptide (e.g., anti-CCAR1 antibodies) and/or one or more fragments (e.g., immunogenic fragments) of a CCAR1 polypeptide to reduce or eliminate the number of cancer cells present within the mammal. For example, the methods and materials described herein can be used to reduce the number of cancer cells present within a mammal having cancer by, for example, 10, 20, 30, 40, 50, 60, 70, 80, 90, 95, or more percent. For example, the methods and materials described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

In some cases, the methods and materials provided herein can be used to improve survival of a mammal (e.g., a human) having cancer. For example, a mammal in need thereof (e.g., a mammal having cancer) can be administered one or more inhibitors of a CCAR1 polypeptide (e.g., anti-CCAR1 antibodies) and/or one or more fragments (e.g., immunogenic fragments) of a CCAR1 polypeptide to improve survival of the mammal. For example, the methods and materials described herein can be used to improve the survival of a mammal having cancer by, for example, 10, 20, 30, 40, 50, 60, 70, 80, 90, 95, or more percent. For example, the methods and materials described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, about 8 years, about 10 years, or more).

In some cases, the methods and materials provided herein can be used to delay or prevent the development of a cancer in a mammal (e.g., a human) at risk of developing cancer. For example, a mammal in need thereof (e.g., a mammal at risk of developing cancer) can be administered one or more inhibitors of a CCAR1 polypeptide (e.g., anti-CCAR1 antibodies) and/or one or more fragments (e.g., immunogenic fragments) of a CCAR1 polypeptide to delay the development of cancer (e.g., a recurrent cancer) by, for example, 10, 20, 30, 40, 50, 60, 70, 80, 90, 95, or more percent. In some cases, the methods and materials described herein can be used to delay the development of cancer (e.g., a recurrent) by at least 6 months (e.g., about 6 months, about 8 months, about 10 months, about 1 year, about 1.5 years, about 2 years, about 2.5 years, about 3 years, about 4 years, about 5 years, about 8 years, about 10 years, or more).

When treating a mammal (e.g., a human) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer, the cancer can be any type of cancer. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a recurrent cancer. Examples of cancers that can be treated as described herein include, without limitation, prostate cancers, breast cancers, leukemia cancers, lung cancers, melanoma, uterine cancers, kidney cancers, thyroid cancers, ovarian cancer, and colon cancer.

In some cases, the methods described herein can include identifying a mammal (e.g., a human having an autoimmune disease such as DM) as having cancer. For example, imaging techniques, biopsy techniques (e.g., liquid biopsies), blood tests, urine tests, and/or genetic tests (e.g., cytogenetics, fluorescent in situ hybridization (“FISH”)) for mutations and tumor markers can be used to identify mammals (e.g., humans) having cancer.

Once a mammal (e.g., a human) having an autoimmune disease (e.g. DM) is identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or is identified as having cancer, the mammal can be treated for cancer. In some cases, a mammal identified as having higher risk for cancer as described herein and/or identified as having cancer can be administered or instructed to self-administer one or more inhibitors of a polypeptide described herein and/or one or more fragments (e.g., immunogenic fragments) of a polypeptide described herein. For example, a mammal identified as having higher risk for cancer as described herein and/or identified as having cancer can be administered or instructed to self-administer one or more inhibitors of a CCAR1 polypeptide (e.g., anti-CCAR1 antibodies) and/or one or more fragments (e.g., immunogenic fragments) of a CCAR1 polypeptide.

An inhibitor of a polypeptide described herein can be an inhibitor of polypeptide activity or an inhibitor of polypeptide expression. For example, an inhibitor of a CCAR1 polypeptide can be an inhibitor of CCAR1 polypeptide activity or an inhibitor of CCAR1 polypeptide expression. Examples of compounds that can reduce or eliminate CCAR1 polypeptide activity include, without limitation, polypeptides that can target (e.g., target and bind to) a CCAR1 polypeptide (e.g., anti-CCAR1 antibodies such as neutralizing anti-CCAR1 antibodies), and small molecules that can target (e.g., can target and bind) to a CCAR1 polypeptide. When a compound that can reduce or eliminate CCAR1 polypeptide activity is a small molecule that targets (e.g., targets and binds) to a CCAR1 polypeptide, the small molecule can be in the form of salt (e.g., a pharmaceutically acceptable salt). Examples of compounds that can reduce or eliminate CCAR1 polypeptide expression include, without limitation, nucleic acid molecules designed to induce RNA interference of BRD4 polypeptide expression (e.g., a siRNA molecule or a shRNA molecule), antisense molecules, and miRNAs.

An anti-CCAR1 antibody can target (e.g., target and bind to) any appropriate CCAR1 polypeptide. Examples of CCAR1 polypeptides that can be targeted by an anti-CCAR1 antibody include, without limitation, those set forth in the National Center for Biotechnology Information (NCBI) databases under Accession Nos. NP_060707 (Version NP_060707.2; GI: 46852388), Q8IX12 (Version Q8IX12.2), AAI30627 (Version AAI30627.1), GI: 545479138, and GI: 545478468.

An anti-RCC1 antibody can target (e.g., target and bind to) any appropriate RCC1 polypeptide. An example of a RCC1 polypeptide that can be targeted by an anti-RCC1 antibody can be, without limitation, the polypeptide set forth in the NCBI databases under Accession No. NP_001041659.1.

An anti-GATD1 antibody can target (e.g., target and bind to) any appropriate GATD1 polypeptide. An example of a GATD1 polypeptide that can be targeted by an anti-GATD1 antibody can be, without limitation, the polypeptide set forth in the NCBI databases under Accession No. NP_001305750.1.

An anti-TBL1XR1 antibody can target (e.g., target and bind to) any appropriate TBL1XR1 polypeptide. An example of a TBL1XR1 polypeptide that can be targeted by an anti-TBL1XR1 antibody can be, without limitation, the polypeptide set forth in the NCBI databases under Accession No. NP_001308122.1.

An anti-KDM2A antibody can target (e.g., target and bind to) any appropriate KDM2A polypeptide. An example of a KDM2A polypeptide that can be targeted by an anti-KDM2A antibody can be, without limitation, the polypeptide set forth in the NCBI databases under Accession No. NP_055828.2.

An anti-IMMT antibody can target (e.g., target and bind to) any appropriate IMMT polypeptide. An example of a IMMT polypeptide that can be targeted by an anti-IMMT antibody can be, without limitation, the polypeptide set forth in the NCBI databases under Accession No. NP_001093639.1.

An anti-SOX5 antibody can target (e.g., target and bind to) any appropriate SOX5 polypeptide. An example of a SOX5 polypeptide that can be targeted by an anti-SOX5 antibody can be, without limitation, the polypeptide set forth in the NCBI databases under Accession No. NP_001248344.1.

An anti-CIZ1 antibody can target (e.g., target and bind to) any appropriate CIZ1 polypeptide. An example of a CIZ1 polypeptide that can be targeted by an anti-CIZ1 antibody can be, without limitation, the polypeptide set forth in the NCBI databases under Accession No. NP_001124488.1.

An anti-NVL2 antibody can target (e.g., target and bind to) any appropriate NVL2 polypeptide. An example of a NVL2 polypeptide that can be targeted by an anti-NVL2 antibody can be, without limitation, the polypeptide set forth in the NCBI databases under Accession No. NP_002524.2.

An anti-NACC1 antibody can target (e.g., target and bind to) any appropriate NACC1 polypeptide. An example of a NACC1 polypeptide that can be targeted by an anti-NACC1 antibody can be, without limitation, the polypeptide set forth in the NCBI databases under Accession No. NP_443108.1.

As used herein, the term “antibody” includes whole antibodies and antibody fragments and derivatives provided that the fragment or derivative maintains the ability to treat cancer (e.g., by inducing an immune response such as an anti-cancer immune response). Examples of antibody fragments include, without limitation, single-chain variable fragments (scFvs), antigen-binding (Fab) fragments (e.g., Fab′ or (Fab)₂), Fv fragments, polyclonal antibodies, monoclonal antibodies, bispecific antibodies, diabodies, and other antibody-like molecules. An antibody, antibody fragment, or antibody derivative can be of any class (e.g., IgG, IgA, IgM). In some cases, an antibody, antibody fragment, or antibody derivative fragment can be chimeric. In some cases, an antibody, antibody fragment, or antibody derivative can be humanized. In some cases, an antibody, antibody fragment, or antibody derivative can be human antibody, antibody fragment, or antibody derivative.

A fragment (e.g., an immunogenic fragment) of a polypeptide described herein can be any polypeptide fragment that, when administered to a mammal (e.g., a human), can induce an immune response (e.g., can induce production of antibodies) against that polypeptide within the mammal. For example, a fragment (e.g., an immunogenic fragment) of a polypeptide that is recognized by a DCR antibody can be any polypeptide fragment that, when administered to a mammal (e.g., a human), can induce an immune response (e.g., can induce production of antibodies) against the polypeptide that is recognized by a DCR antibody within the mammal. Examples of immunogenic polypeptide fragments that can induce an immune response against a polypeptide that is recognized by a DCR antibody can include, without limitation, the polypeptide fragments set forth in Table 1.

In some cases, one or more inhibitors of a polypeptide described herein and/or one or more fragments (e.g., immunogenic fragments) of a polypeptide described herein can be formulated into a composition (e.g., a pharmaceutically acceptable composition). For example, one or more inhibitors of a CCAR1 polypeptide (e.g., anti-CCAR1 antibodies) and/or one or more fragments (e.g., immunogenic fragments) of a CCAR1 polypeptide can be formulated into a composition (e.g., a pharmaceutically acceptable composition) for administration to a mammal (e.g., a human) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer. For example, one or more inhibitors of a CCAR1 polypeptide (e.g., anti-CCAR1 antibodies) and/or one or more fragments (e.g., immunogenic fragments) of a CCAR1 polypeptide can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, one or more inhibitors of a polypeptide described herein and/or one or more fragments (e.g., immunogenic fragments) of a polypeptide described herein can be formulated for a particular method of administration to a mammal (e.g., a human). For example, a composition (e.g., a pharmaceutically acceptable composition) containing one or more inhibitors of a CCAR1 polypeptide (e.g., anti-CCAR1 antibodies) can be designed for oral or parenteral (including, without limitation, a subcutaneous, intramuscular, intravenous, intradermal, intra-cerebral, intrathecal, or intraperitoneal (i.p.) injection) administration to a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition (e.g., a pharmaceutically acceptable composition) containing one or more inhibitors of a polypeptide described herein and/or one or more fragments (e.g., immunogenic fragments) of a polypeptide described herein can be administered to a mammal (e.g., a human) in any appropriate amount. For example, composition (e.g., a pharmaceutically acceptable composition) containing one or more inhibitors of a CCAR1 polypeptide (e.g., anti-CCAR1 antibodies) can be administered to a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer in any appropriate amount (e.g., any appropriate dose). An effective amount of a composition containing one or more inhibitors of a CCAR1 polypeptide (e.g., anti-CCAR1 antibodies) can be any amount that can treat a mammal identified as having higher risk for cancer as described herein and/or identified as having cancer as described herein without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer in the mammal being treated may require an increase or decrease in the actual effective amount administered.

A composition (e.g., a pharmaceutically acceptable composition) containing one or more inhibitors of a polypeptide described herein and/or one or more fragments (e.g., immunogenic fragments) of a polypeptide described herein can be administered to a mammal (e.g., a human) in any appropriate frequency. For example, composition (e.g., a pharmaceutically acceptable composition) containing one or more inhibitors of a CCAR1 polypeptide (e.g., anti-CCAR1 antibodies) can be administered to a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer in any appropriate frequency. The frequency of administration can be any frequency that can treat a mammal identified as having higher risk for cancer as described herein and/or identified as having cancer without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition (e.g., a pharmaceutically acceptable composition) containing one or more inhibitors of a polypeptide described herein and/or one or more fragments (e.g., immunogenic fragments) of a polypeptide described herein can be administered to a mammal (e.g., a human) for any appropriate duration. For example, composition (e.g., a pharmaceutically acceptable composition) containing one or more inhibitors of a CCAR1 polypeptide (e.g., anti-CCAR1 antibodies) can be administered to a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer for any appropriate duration. An effective duration for administering or using a composition containing one or more inhibitors of a CCAR1 polypeptide (e.g., anti-CCAR1 antibodies) can be any duration that can treat a mammal having, or at risk of developing, cancer without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human having an autoimmune disease such as DM) cancer can include administering to the mammal one or more inhibitors of a polypeptide described herein and/or one or more fragments (e.g., immunogenic fragments) of a polypeptide described herein as the sole active ingredient(s) to treat the mammal. For example, methods for treating a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer can include administering to the mammal one or more inhibitors of a CCAR1 polypeptide (e.g., anti-CCAR1 antibodies) as the sole active ingredient(s) to treat the mammal. For example, a composition (e.g., a pharmaceutically acceptable composition) containing one or more inhibitors of a CCAR1 polypeptide (e.g., anti-CCAR1 antibodies) can include the one or more inhibitors of a CCAR1 polypeptide as the sole active ingredient(s) in the composition that is/are effective to treat a mammal identified as having higher risk for cancer as described herein and/or identified as having cancer.

In some cases, methods for treating a mammal (e.g., a human having an autoimmune disease such as DM) cancer can include administering to the mammal one or more inhibitors of a polypeptide described herein and/or one or more fragments (e.g., immunogenic fragments) of a polypeptide described herein together with one or more (e.g., one, two, three, four, five or more) additional agents/therapies used to treat cancer. For example, methods for treating a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer as described herein (e.g., by administering one or more inhibitors of a CCAR1 polypeptide such as anti-CCAR1 antibodies) also can include administering to the mammal one or more (e.g., one, two, three, four, five or more) additional agents/therapies used to treat cancer. For example, a combination therapy used to treat cancer can include administering to the mammal (e.g., a human) one or more inhibitors of a CCAR1 polypeptide (e.g., anti-CCAR1 antibodies) and one or more (e.g., one, two, three, four, five or more) agents used to treat cancer. Examples of agents that can be administered to a mammal to treat cancer include, without limitation, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, and any combinations thereof. In cases where one or more inhibitors of a CCAR1 polypeptide (e.g., anti-CCAR1 antibodies) are used in combination with additional agents used to treat cancer, the one or more additional agents can be administered at the same time (e.g., in a single composition containing both one or more inhibitors of a CCAR1 polypeptide and the one or more additional agents) or independently. For example, one or more inhibitors of a CCAR1 polypeptide (e.g., anti-CCAR1 antibodies) can be administered first, and the one or more additional agents administered second, or vice versa.

In some cases, methods for treating a mammal (e.g., a human having an autoimmune disease such as DM) cancer can include administering to the mammal one or more inhibitors of a polypeptide described herein and/or one or more fragments (e.g., immunogenic fragments) of a polypeptide described herein together with one or more (e.g., one, two, three, four, five or more) additional therapies used to treat cancer. For example, methods for treating a mammal (e.g., a human having an autoimmune disease such as DM) identified as having higher risk for cancer as described herein (e.g., based, at least in part, on the presence of anti-TIF1-γ antibodies and the absence of a DCR autoantibody described herein (e.g., the absence of any of the DCR antibodies described herein) in a sample obtained from a mammal) and/or identified as having cancer as described herein (e.g., by administering one or more inhibitors of a CCAR1 polypeptide such as anti-CCAR1 antibodies) also can include subjecting the mammal to one or more (e.g., one, two, three, four, five or more) additional therapies used to treat cancer. Examples of therapies used to treat cancer include, without limitation, surgery, and/or radiation therapy. In cases where one or more inhibitors of a CCAR1 polypeptide (e.g., anti-CCAR1 antibodies) are used in combination with one or more additional therapies used to treat cancer, the one or more additional therapies can be performed at the same time or independently of the administration of one or more inhibitors of a CCAR1 polypeptide (e.g., anti-CCAR1 antibodies). For example, one or more inhibitors of a CCAR1 polypeptide (e.g., anti-CCAR1 antibodies) can be administered before, during, or after the one or more additional therapies are performed.

The invention will be further described in the following examples, which do not limit the scope of the invention described in the claims.

### EXAMPLES

### Example 1: Immune Responses to CCAR1 and Other Novel Dermatomyositis Autoantigens were Associated with Attenuated Cancer Emergence

This example identifies autoantibodies against cell division cycle and apoptosis regulator protein 1 (CCAR1) present in dermatomyositis (DM) patients. The presence of anti-CCAR1 autoantibodies is associated with a decrease in cancer emergence within 3 years in DM patients. Cancers that did emerge occurred after DM onset and were more likely to be localized. Additional novel autoantibodies defined were similarly associated with decreased frequency of cancer diagnosis.

### Materials and Methods

### Study Design

The objective of this study was to identify autoantigens preferentially targeted by patients in whom cancer did not emerge, utilizing a high cancer-risk DM population (defined as having anti-TIF1-γ antibodies). This was a cohort design in which the study population consisted of DM patients who consented to donate blood, and who were found to have anti-TIF1-γ autoantibodies in their serum.

### Discovery Cohort

Patients were considered to have DM if they met Bohan and Peter criteria for DM, or, for clinically amyopathic patients, the investigator (DFF) considered their rash and skin biopsy to be consistent with DM (see, for example, Bohan A, and Peter J B. N Engl J Med. 1975; 292(7):344-7). All patients had onset of DM after 18 years of age. Clinical data were abstracted from the study database. DM onset was defined as either the date of first rash or muscle weakness, whichever came first.

### Validation Cohort

All participants in the study met the definition of probable or definite DM by Bohan and Peter criteria. To capture patients with clinically amyopathic DM, patients with Gottron's and/or Heliotrope sign with interface dermatitis on skin biopsy were also included. All patients had onset of DM after 18 years of age. Clinical data were abstracted from the study database and from the electronic medical record. DM onset was defined as first symptom as reported by patient including rash, weakness, myalgia, or dyspnea.

### Healthy Controls

Serum was obtained from 34 healthy control subjects.

### Scleroderma Cohort

This cohort consisted of sera from 68 well-characterized scleroderma patients with anti-POL3A antibodies. Thirty-four sera were from patients with a history of cancer, and 34 were from patients who had no history of cancer after at least 5 years of follow-up.

### Cancer Screening and Definitions

Timing and methodology for cancer screening was determined by the treating physician. The vast majority of patients received computed tomography scanning of chest, abdomen, and pelvis at least once during the first 3 years following DM onset, in addition to age and sex appropriate cancer screening. Cancer was defined as any malignancy diagnosed with tissue biopsy, excluding non-melanoma cancer of the skin. The American Joint Committee on Cancer staging classification system was used to define stage of cancer at the cancer index date. Cancer index date was defined as the date of cancer diagnosis, or, in cases where the cancer was in clinical remission and later recurred, either the date of recurrence or original diagnosis was used, whichever was closest to date of DM symptom onset.

### Cell Cultures and Immunoblotting

HeLa, A431 and 624 melanoma cells were cultured using standard tissue culture procedures. For immunoblotting, A431 and HeLa cells were washed extensively with PBS before lysing with buffer A (1% Nonidet P40 [NP40], 20 mM Tris [pH 7.4], 150 mM NaCl, 1 mM EDTA, and a protease inhibitor cocktail). Cell lysates (20 micrograms/lane for the TIF1-γ blots and 5 micrograms/lane for the CCAR1 blots) were electrophoresed on 10% SDS-polyacrylamide gels, and then transferred to nitrocellulose membranes. Immunoblots were performed using a rabbit polyclonal anti-CCAR1 antibody (Novus Biologicals, 1:7,500 dilution) or a mouse monoclonal anti-TIF1-γ antibody (Novus Biologicals, 1:1,000 dilution), followed by incubation with horseradish-peroxidase-labeled secondary antibodies (Pierce, Rockford, IL) and chemiluminescence. Images were acquired using a Protein Simple Fluorochem-M digital imager.

### Sample Collection and TIF1-γ Antibody ELISA Assay

Plasma/serum was obtained from all DM patients from both cohorts on (or within 6 months) the date of their initial clinic visit and aliquots were banked at −80° C. The same sample was used for all autoantibody testing in the study. Antibodies against TIF1-γ were determined by ELISA using a commercially available ELISA kit (MBL, Japan). The cutoff for antibody positivity was set at 7 units; this value was based on the mean+4 SD of values obtained from 67 healthy controls that were assayed with this kit. Of note, a comparison of the TIF1-γ antibody status obtained using this ELISA compared to those obtained with an IP/immunoblot (IP/blot) assay gave similar results overall, with the ELISA assay being more sensitive. The ELISA was able to detect TIF1-γ antibodies at 7 units, while the lower limit for detection with IP/blot was in the 10-15 unit range.

### Immunoprecipitation (IP) Using 35S-Methionine-Labeled In Vitro Transcription Translation (IVTT) Proteins to Detect Antibodies (IVTT-IP)

Complementary DNAs (cDNAs) encoding full-length FLAG-tagged human CCAR1, RCC1, GATD1, TBL1XR1, KDM2A, IMMT, SOX5, CIZ1, NVL2 and NACC1 were purchased from Genscript (Piscataway, NJ). All DNAs were sequence verified before use. 35S-methionine-labeled proteins were generated from these cDNAs by IVTT reactions, per the manufacturer's protocol (Promega). IPs performed using these products as input material were electrophoresed on 10% SDS-PAGE gels and visualized by fluorography. IPs performed with a positive reference serum (anti-SOX5 and anti-CIZ1), or an anti-FLAG IP (all other IVTT products) were included in each sample set, and fluorogram exposures were standardized to give reference IP bands a similar intensity. Positive/negative antibody status was assigned by independent visual inspection of the equivalently exposed autorads by 2 skilled investigators (LCR and AR). All samples assigned a positive antibody status (and a subset of the negative samples) were assayed a second time to confirm positivity. Sera from healthy controls were also tested by IP with each of the IVTT products. No IP band was detected with any of the control sera.

Immunoprecipitations Performed from Radiolabeled 624 Melanoma Cell Lysates

624 melanoma cells were radiolabeled with 35S-methionine and used for IPs performed with patient plasma. The IPs were electrophoresed on 10% SDS-polyacrylamide gels and visualized by fluorography. An IP performed with the same anti-PMSCL reference serum was included in each set to standardize exposure intensities.

### Computational Analysis of IP Traces

Fluorograms of electrophoresed IPs (equivalently exposed based on the intensity of the anti-PMSCL reference IP bands) were scanned by densitometry (BioRad software), producing a vector of absorbance values for each patient, with higher numbers corresponding to darker fluorogram bands. Comparability of absorbance values across patients was ensured by first smoothing with a Gaussian filter of width 1 (to account for noise in the densitometer's reads), and aligning to the TIF1-γ peak (the first and highest peak on each trace, at ˜ 0.1 relative front). The mean across patients in each group was then computed separately, and the standard error of the mean at each point (shown as confidence intervals) was computed using a bootstrapping procedure. Bands were distinguished by applying a standard peak-finding procedure (implemented in Scipy's signal package) to the smoothed vector of absorbance values. In each serum, peaks were sorted by their amplitude (absorbance) and expressed as a fraction of that serum's TIF1-γ absorbance. These values were used to compute the mean number of bands that were between 0 and 100 percent as high as the TIF1-γ peak for each disease subgroup, as well as the standard error of the subgroup sample mean at each point.

### Identification of New Antibody Specificities by Mass Spectrometry

IPs were performed as described above, using lysates made from unlabeled 624 melanoma cells and selected plasma samples from patients without cancer. The amount of lysate and plasma used per IP for these assays was scaled up 5-fold relative to the radiolabeled IPs. On-bead digests were performed with trypsin/LysC and the resulting peptides were analyzed by reverse phase LC-MS. Eluting peptides were sprayed into a Q-Exactive Plus (QE Plus, Thermo Scientific) mass spectrometer. Isotopically resolved masses were extracted using Proteome Discoverer software and searched using Mascot 2.5.1 through Proteome Discoverer against a human protein database. Peptide identifications from Mascot searches were processed within Scaffold (Proteome Software) with display criteria set to 95% confidence for both protein and peptide identifications.

### Co-IP of TIF1-g and CCAR1

HSG cells were treated with 50 mM etoposide (Cell Signaling) for 3 hours, then washed with PBS on ice and lysed in RIPA buffer (50 mM Tris pH 7.4, 150 mM NaCl, 5 mM EDTA, 0.5% Nonidet P40, 0.5% sodium deoxycholate, 0.1% SDS, with phosphatase inhibitors and protease inhibitors added). After centrifugation at 16,000 rpm (20 minutes, 4° C.), the supernatants were diluted in Buffer A, precleared with Protein A beads and then used as input for IPs. These were performed by incubating with (i) an anti-CCAR1 rabbit polyclonal antibody (Novus Biologicals) or (ii) an anti-TIF1-γ rabbit monoclonal antibody (Cell Signaling) for 90 minutes at 4° C., followed by addition of Protein A agarose beads (25 minutes, 4° C.). Control IPs were performed by omitting the primary antibody, and incubating with Protein A beads only. After extensive gentle washing, the IPs were electrophoresed on 8% SDS-polyacrylamide gels and transferred to nitrocellulose membranes. Immunoblots of the IPs were performed using a mouse monoclonal anti-TIF1-γ antibody or a rabbit polyclonal anti-CCAR1 antibody (for IPs performed with anti-CCAR1 or anti-TIF1-Y, respectively) as described above.

### Statistics

All analyses were performed using Stata version 14 (College Station, TX). Logistic regression and Fischer exact testing were used to assess associations between CCAR1 autoantibodies and cancer. Differences between continuous variables were summarized and significance analyzed using a t-test or Mann-Whitney test (normally vs not normally distributed variables, respectively).

### Results

Autoantibody Response in Anti-TIF1-γ-Positive DM Patients that do not Develop Cancer.

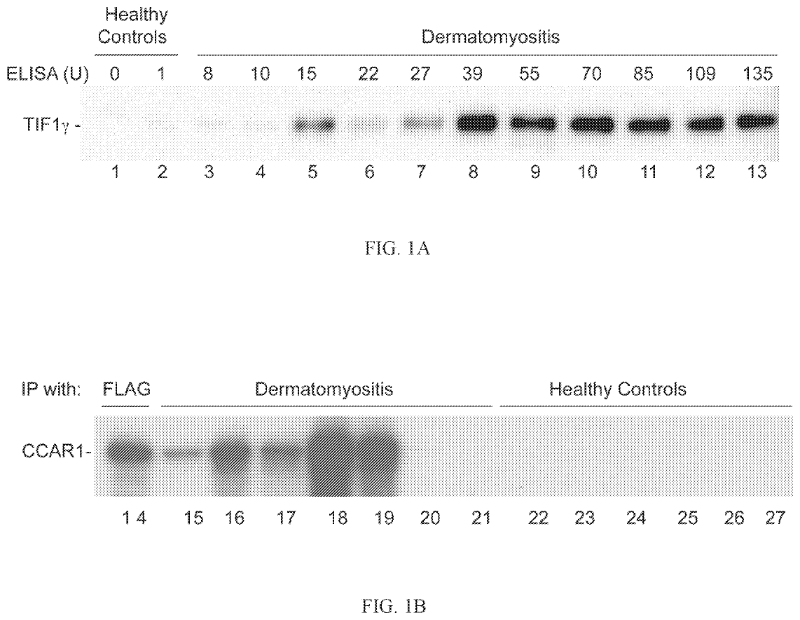

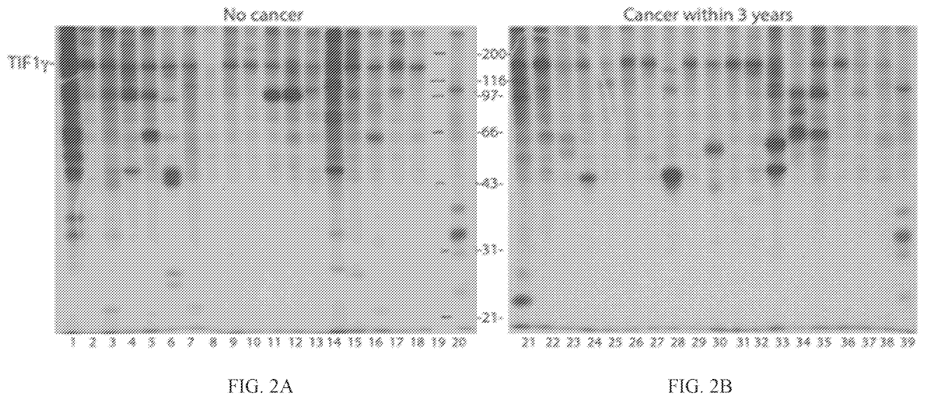

This study was designed to identify novel autoantibodies associated with infrequent emergence of cancer in a DM population with high cancer risk. The analysis was restricted to patients with antibodies against TIF1-γ. ELISA and IP/blot assays were compared for their ability to detect anti-TIF1-γ antibodies (FIG. 1A-1B). An immunoprecipitation (IP) approach was initially used. 36 DM patients with anti-TIF1-γ antibodies were selected from the discovery cohort. Of these 36 DM patients with anti-TIF1-γ antibodies, 18 had a cancer diagnosed within 3 years of DM symptom onset, and 18 had no malignancy detected with at least 3 years of follow-up from DM symptom onset. Plasma from these patients was used to IP proteins from radiolabeled cell lysates, and equivalently exposed fluorograms were examined to compare the IP patterns (FIG. 2A-2B).

Six hundred twenty-four melanoma cells were radiolabeled with 35S-methionine and used for IPs performed with plasma from 36 anti-TIF1-γ-positive DM patients. Samples were from patients without (FIG. 2A) or with (FIG. 2B) a cancer detected within 3 years of DM diagnosis. In each set, an IP performed with the same anti-PMSCL-positive patient serum (lanes 20 and 39) was included. This was done to facilitate equivalent exposures of the fluorograms based on the reference IP band intensities. Migration of molecular weight standards were marked between the panels, and are shown in lane 19. A 155 kDa band (TIF1-γ) was commonly detected as a prominent band in both sets (with and without cancer). Visual inspection of the IP data revealed additional autoantibody specificities in the group without cancer.

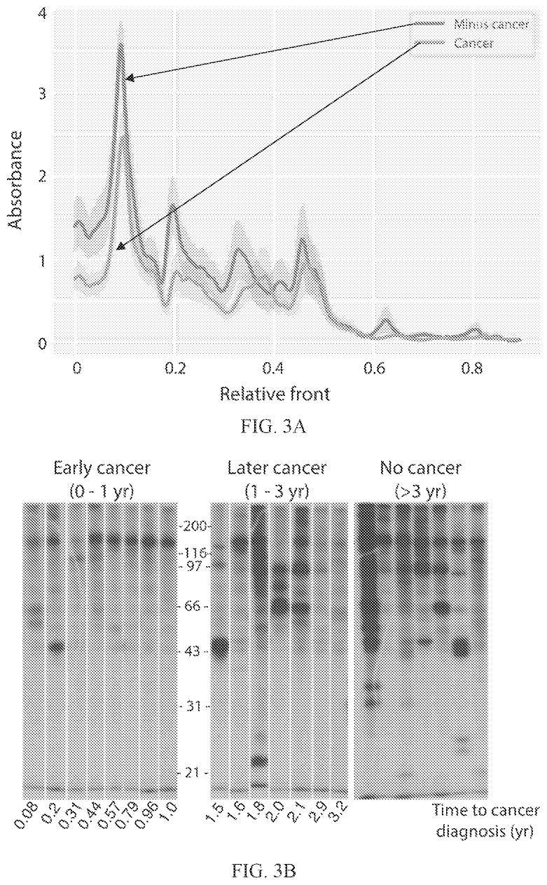

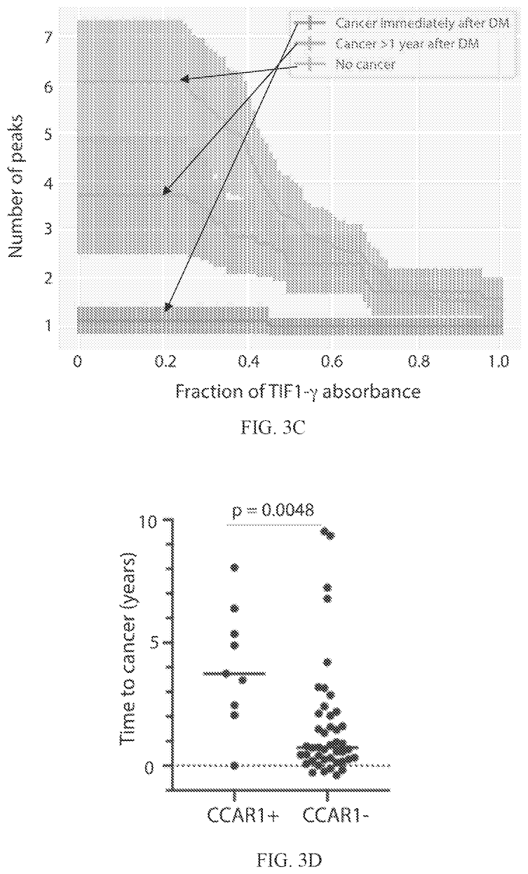

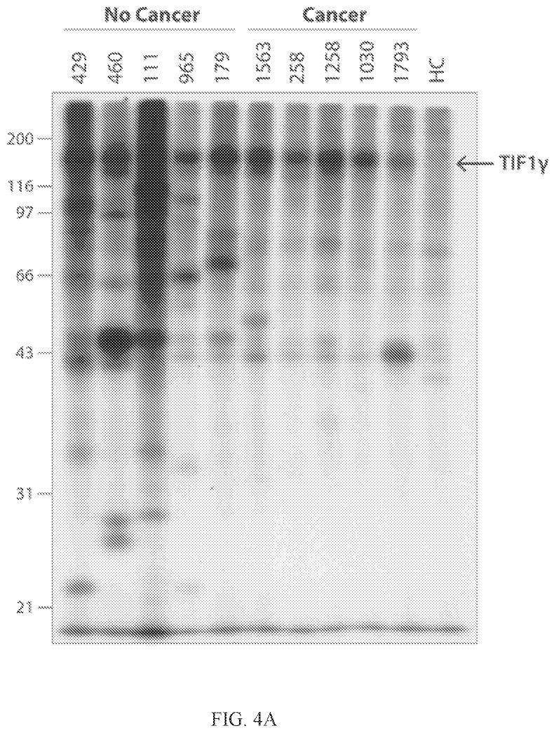

IP data were quantitated using a signal processing analysis, which calculated mean densitometric values at any given distance of migration along the gel. The resulting mean IP traces for the cancer versus non-cancer groups confirmed that at several areas (corresponding to molecular weights approximately 20-25 kDa, 50-60 kDa, and 85-95 kDa) the non-cancer patients had a relatively higher mean density of immunoprecipitated material (FIG. 3A). This result suggested that cancer-negative DM patients in general have a relatively larger number of prominent autoantibody specificities.

The raw IP data was visualized by digitally arranging the gel lanes in order (left-to-right) from short interval cancer (<1 year), longer interval cancer (1-3 year), and no cancer. An increasing number of immunoprecipitated targets, moving from short-interval cancer to long-interval cancer to no cancer, was observed (FIG. 3B). Gel lanes from FIG. 3B were selected for presentation as follows: all samples from the left panel were selected if the cancers occurred at the time of, or after, DM onset. In addition, lanes 1-7 on the “No Cancer” gel were selected. Lanes from the patients with cancer were digitally rearranged and shown in increasing order of time interval between DM onset and cancer diagnosis.

The number of antibodies in each IP was quantified by calculating the average number of bands relative to their intensity in each serum. In FIG. 3C, antibody diversity is shown for each patient subgroup, as quantified by the number of absorbance peaks as a function of magnitude relative to the TIF1-γ peak. This method captures antibody diversity that corresponds with absorbance peaks of progressively higher amplitude as x-values increase. The autoantibody response was more exclusively focused on TIF1-γ in patients where cancer emerges <1 year of DM diagnosis, compared to the no-cancer subgroup, at both low and high amplitudes, which shows a broader set of autoantibody specificities. DM patients where cancer appears after 1 year have an intermediate breadth of autoantibody focus. Using this approach, the short-interval cancer group had significantly fewer detectable immunoprecipitated specificities for all intensities than the long-interval cancer group, which, in turn, had fewer than the non-cancer group (FIG. 3C).

In FIG. 3D, the distribution of delay of cancer diagnosis relative to DM onset is shown for anti-TIF1-γ-positive DM patients with and without anti-CCAR1 antibodies. All anti-TIF1-γ-positive patients (combined cohorts) with cancers diagnosed between 0 and 10 years after DM onset are shown. Median values for each patient group are indicated by horizontal bars with p value for difference in medians shown (Mann-Whitney test, two-tailed).

Discovery of Novel Autoantigens in Anti-TIF1-γ-Positive DM Patients without Cancer

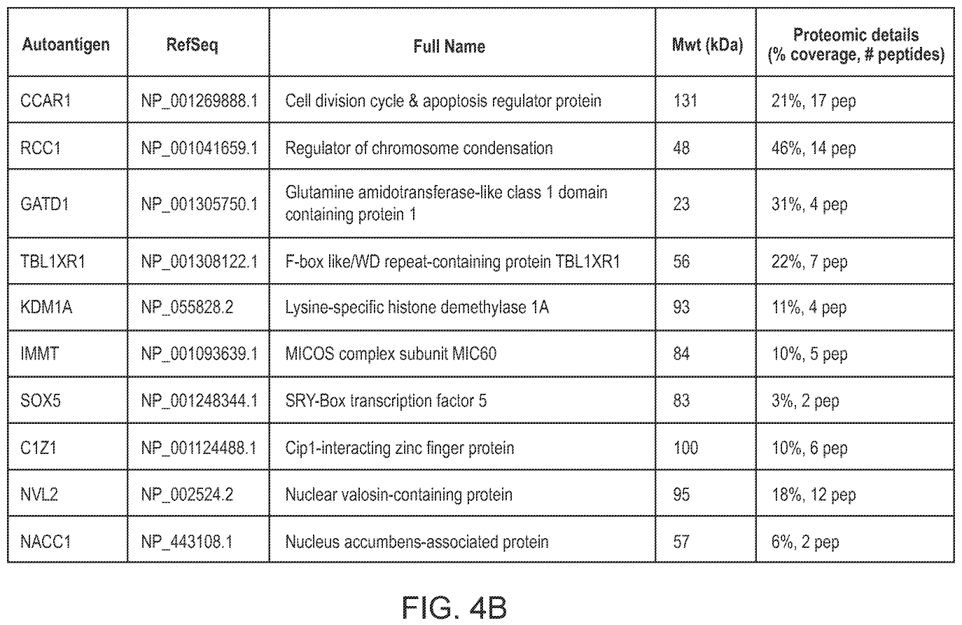

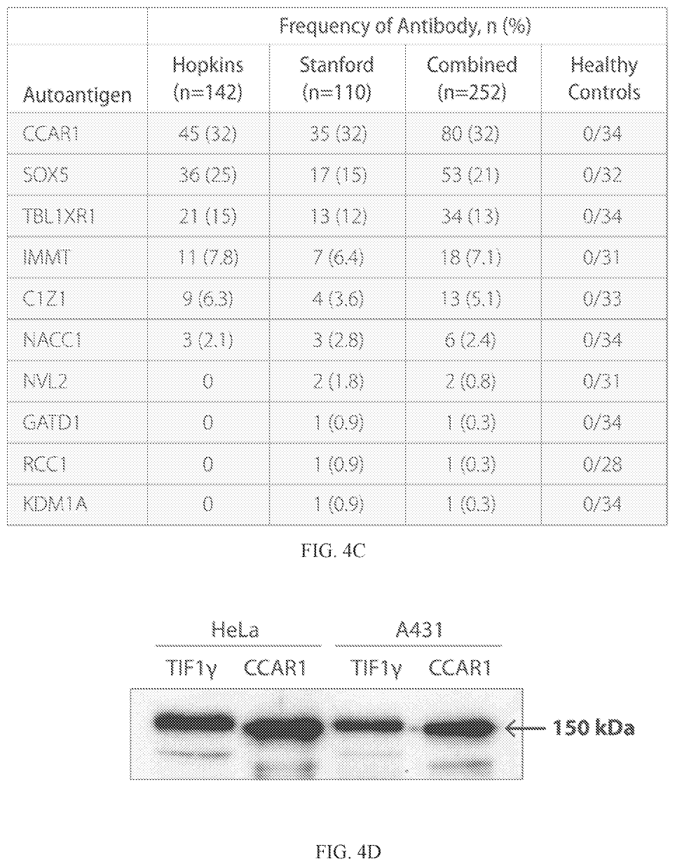

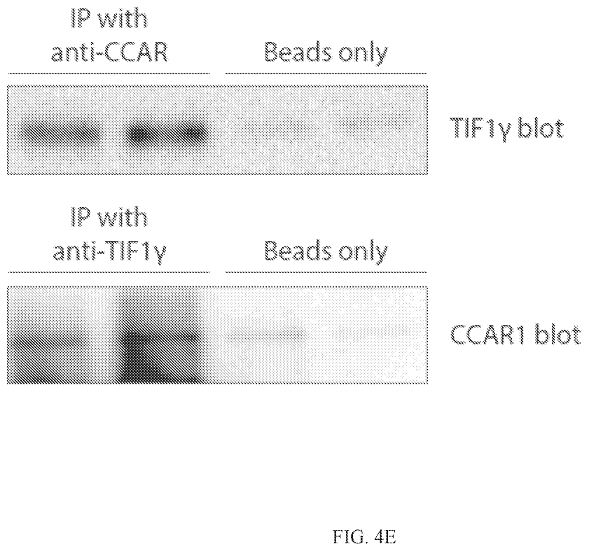

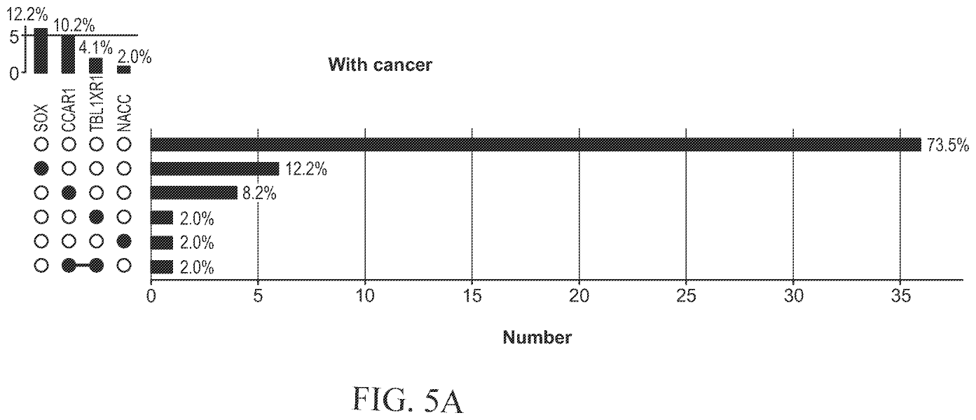

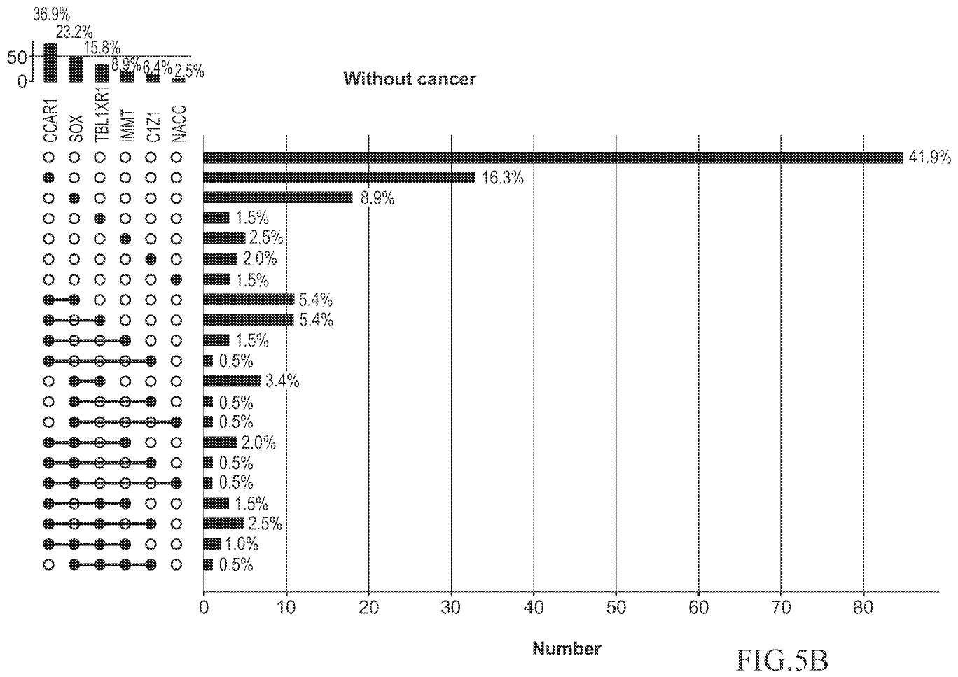

For discovery of these autoantigens, plasma from 5 DM patients from this group of 18 for additional study was selected based on the most prominent IP patterns. The IP profiles of the 5 selected samples are shown in FIG. 4A. IPs using 5 samples from the anti-TIF1-γ-positive DM group with cancer were included for comparison. IPs from the 5 DM patients without cancer were subjected to mass spectrometry (MS)-sequencing, which identified multiple novel putative autoantigens targeted by each sample. Of the list of 23 possible candidates that were generated, 13 were prioritized for validation based on greatest percent coverage and availability of validation reagents. An IP performed with a sample from a healthy control individual is shown in the right-most lane. Migration of molecular weight standards were marked on the left. Validation of these putative autoantigens was performed by IP using 35S-methionine labeled proteins generated by IVTT and the relevant index serum sample in each case.

Of these putative autoantigens, three (anti-ADNP, anti-RAN, and anti-CPSF6) were not immunoprecipitated by their index serum, while ten autoantigens were validated (FIG. 4B). Three patients without cancer each had a single new specificity identified (anti-CCAR1, anti-NVL2, and anti-NACC1), one patient had 2 new specificities identified (anti-RCC1 and anti-GATD1) and one patient (patient 111) had 5 new antibody specificities identified (anti-TBL1XR1, anti-KDM2A, anti-IMMT, anti-SOX5, and anti-CIZ1). Sera from healthy controls did not have antibodies against any of these antigens (FIG. 4C). The prevalence of each of the 10 validated new specificities was determined by IVTT-IP assay in the discovery cohort, which included 110 anti-TIF1-γ positive DM patients. Of these, antibodies against CCAR1 (anti-CCAR1) were the most frequent (detected in 35/110 (32%) of patients (FIG. 4C). The other novel specificities were found with frequencies ranging from 0.9-15%. Anti-CCAR1 was therefore prioritized for initial studies to investigate whether the presence of additional antibodies influenced the frequency of cancer diagnosis in anti-TIF1-γ positive DM patients. Since CCAR1 and TIF1-γ have similar molecular weights and co-migrate (FIG. 3D), these two specificities cannot be accurately distinguished on the autoradiogram patterns in IPs performed on radiolabeled lysates.

Enrichment of Anti-CCAR1 Autoantibodies in 2 Independent Cohorts of Anti-TIF1-γ-Positive DM Patients without Cancer

The frequency of novel autoantibodies in an independent validation cohort of anti-TIF1-γ-positive patients was evaluated (n=142). Demographic and clinical characteristics of patients in both cohorts are shown in Table 3. The majority of patients in both cohorts were Caucasian females. The discovery included patients who were slightly older compared to the validation cohort (mean age±standard deviation (SD) of 51±16 vs 45±5). The discovery cohort had a larger Hispanic population (21% vs 5%). The disease duration for patients designated cancer-negative was at least 3 years, but on average was greater than 9 years for both cohorts (9.7 and 9.5 years for the discovery and validation cohorts, respectively). The cohorts were serologically remarkably similar, with anti-CCAR1 autoantibodies present in 32% of patients in both and had similar cancer prevalence: 38 (35%) patients developed a cancer in the discovery cohort, 22 (20%) of which were within 3 years from DM-symptom onset. Similarly, a total of 44 (31%) patients developed a cancer in the validation cohort, 27 (19%) of which were within 3 years of DM-symptom onset. Anti-CCAR1 antibodies were very rare in patients that are negative for anti-TIF1-γ antibodies. Of 172 anti-TIF1-γ negative patients in the discovery DM cohort, only 1 (0.6%) tested positive for anti-CCAR1 antibodies. This patient also had antibodies against MDA5, NXP2, TBLX and SOX5.

TABLE 3
Demographics and clinical characteristics of the discovery
dermatomyositis and validation myositis cohorts.

| | discovery | | validation | |
|---|---|---|---|---|
| | n = 110 | | n = 142 | |
| Demographic^{A} and Clinical Characteristics | N (%) | | N (%) | |
| Age at DM symptom onset (mean ± SD^{B}) | 51 ± 16 | | 45 ± 5 | |
| Female | 88 | (80) | 117 | (82) |
| Race | | | | |
| Caucasian | 86 | (78) | 127 | (89) |
| African American | 3 | (3) | 10 | (7) |
| Asian | 8 | (7) | 3 | (2) |
| American Indian or Alaskan Native | 0 | | 2 | (2) |
| Native Hawaiian or other Pacific Islander | 1 | (1) | 0 | |
| Other | 2 | (2) | 0 | |
| Unknown | 10 | (9) | 0 | |
| Ethnicity | | | | |
| Hispanic | 23 | (21) | 7 | (5) |
| Non-Hispanic | 76 | (69) | 122 | (86) |
| Unknown | 11 | (10) | 13 | (9) |
| Disease Duration, years, mean (range) | 9.7 (3.1- | | 9.5 (3.2- | |
| | 24.7) | | 26.4) | |
| Serologic Characteristics | | | | |
| Anti-TIF1-γ | 110 | (100) | 142 | (100) |
| Anti-CCAR1 | 35 | (32) | 45 | (32) |
| Cancer Prevalence | | | | |
| Cancer (ever) | 38 | (35) | 44 | (31) |
| Cancer ±5 years of DM symptom onset | 28 | (25) | 30 | (21) |
| Cancer ±3 years of DM symptom onset | 22 | (20) | 27 | (19) |

^{A}Race and ethnicity were patient-reported.
^{B}SD, standard deviation

Within the validation cohort, there was an association between anti-CCAR1 positivity and younger age of DM symptom onset (median 44.0 vs 46.5, rank sum p=0.026), as well as a higher anti-CCAR1 prevalence in Caucasian patients (35% Caucasians positive for anti-CCAR1 vs 7% non-Caucasian, Fischer exact p=0.037). Similar associations were found within the discovery cohort with regards to a younger age of DM onset (median 47.0 vs 49.8, rank sum p=0.49) and higher prevalence in Caucasians (35% Caucasians positive for CCAR1 vs 0% non-Caucasians, Fischer exact p=0.009). Whereas in the discovery cohort there was an association between female sex and anti-CCAR1-positive status (39% women were anti-CCAR1-positive vs 5% of men, Fischer exact p=0.002), this was not replicated in the validation cohort (33% women were anti-CCAR1-positive vs 28% of men, Fischer exact p=0.814). In neither cohort was an association between anti-CCAR1 and any specific cancer type present.

To address whether anti-CCAR1 antibodies are uniquely found in DM patients, or are also found in other autoimmune diseases known to have an association with malignancy, 68 sera from anti-POLR3A-positive scleroderma patients were assayed. These included 34 sera from patients with a history of cancer and 34 who had no history of cancer after at least 5 years of follow-up. Anti-CCAR1 antibodies were found in only 1/68 sera (1.5%) in this cohort. The anti-POLR3Apositive/anti-CCAR1-positive patient had no detected cancer, and it is noteworthy that levels of anti-CCAR1 antibodies were very low in this serum.

The relationship between anti-CCAR1 antibodies and cancer in anti-TIF1-γ-positive DM patients is shown in Table 4. In the discovery Cohort, anti-CCAR1 autoantibodies were significantly negatively associated with a diagnosis of cancer within 3 years of first DM-symptom, OR 0.27 (95% CI 0.7-1.00), p=0.050. Similarly, in the validation cohort, anti-CCAR1 autoantibodies were significantly negatively associated with a history of cancer within 3 years, OR 0.13, (95% CI 0.03-0.59), p=0.008. A sensitivity analysis was performed to minimize the potential impact of immortal person-time bias, in which cancers preceding DM symptom-onset were excluded. The results were unchanged (Table 5). In addition, the negative cancer association with anti-CCAR1 autoantibodies persisted even after controlling for potential confounders (age and biological sex) in multivariable analyses (validation OR 0.13, 95% CI 0.029-0.58, p=0.008; discovery OR 0.24, 95% CI 0.06-0.99, p=0.049).

TABLE 4
The association between anti-CCAR1 antibodies and cancer prevalence
in anti-TIF1-γ-positive dermatomyositis patients.

| | | discovery | | validation | |
|---|---|---|---|---|---|
| | Cancer | | p- | | p- |
| | Window | OR (95% CI) | value | OR (95% CI) | value |
| Anti- | Ever | 0.44 (0.18-1.10) | 0.082 | 0.53 (0.23-1.20) | 0.127 |
| CCAR1 | ±5 Years | 0.49 (0.18-1.36) | 0.177 | 0.11 (0.03-0.50) | 0.004 |
| | ±3 Years | 0.27 (0.07-1.00) | 0.050 | 0.13 (0.03-0.59) | 0.008 |

Analysis includes cancers diagnosed both before and after DM-symptom onset.
OR, odds ratio;
CI, 95% confidence interval

TABLE 5
The effect of anti-CCAR1 on cancer prevalence in
anti-TIF1-γ-positive dermatomyositis patients.

| | | discovery | | validation | |
|---|---|---|---|---|---|
| | Cancer | | p- | | p- |
| | Window | OR (95% CI) | value | OR (95% CI) | value |
| Anti- | Ever | 0.28 (0.08-0.89) | 0.031 | 0.54 (0.20-1.47) | 0.228 |
| CCAR1 | Within | 0.45 (0.14-1.48) | 0.190 | 0.09 (0.01-0.70) | 0.022 |
| | 5 Years | | | | |
| | Within | 0.12 (0.02-0.95) | 0.045 | 0.09 (0.01-0.76) | 0.026 |
| | 3 Years | | | | |

Analysis includes cancers diagnosed only after DM-symptom onset.

### A Physical Complex Containing CCAR1 and TIF1-γ

As noted above, antibodies against CCAR1 were restricted to the population with anti-TIF1-γ antibodies (only 1/172 anti-TIF1-γ-negative patients in the discovery cohort had antibodies against CCAR1). This near-perfect association of the presence of anti-CCAR1 antibodies with concomitant antibodies against TIF1-γ suggested that an underlying mechanism driving this finding might be intermolecular epitope spreading, generally the result of association of the two antigens in a molecular complex. Whether CCAR1 and TIF1-γ exist in a complex was therefore tested. IPs performed from cell lysates using polyclonal rabbit anti-CCAR1 antibodies contained TIF1-γ (FIG. 4E, upper panel). In a reciprocal strategy, CCAR1 was found to be present in IPs done from cell lysates using a rabbit monocloncal anti-TIF1-γ (FIG. 4E, lower panel), together demonstrating that these molecules are found in the same complex.

Later Appearance and Less Advanced Stage in Cancers from Anti-CCAR1-Positive Patients

The timing of cancer diagnosis and stage of those cancers diagnosed in anti-TIF1-γ only DM patients was investigated (10 patients in the validation cohort, 8 patients in the discovery cohort). The timing of cancer appearance (relative to DM onset) and the cancer stage of all 82 patients with malignancies were recorded (Table 6). The similarity in clinical evaluation, data collection, and consistency of the anti-CCAR1-positive associations in the two cohorts allowed the cohorts to be pooled. Cases in which cancer preceded DM onset, or emerged >10 years after DM onset were excluded.

TABLE 6
Full list of all 82 cancers from both
validation and discovery Cohorts.

| | Time from | Cancer | |
|---|---|---|---|
| | Cancer to DM | Stage Upon | Autoantibody |
| Cancer Type | Onset (years) | Diagnosis | Status |
| Prostate | −13.2 | . | CCAR1 |
| Breast | −12.4 | . | CCAR1 |
| Leukemia | −10.96 | . | CCAR1 |
| Breast | −10.9 | 1 | CCAR1 |
| Lung | −6.96 | 3 | CCAR1 |
| Melanoma | −6.03 | 0 | CCAR1 |
| Breast | −0.917 | 2 | CCAR1 |
| Breast | −0.88 | 2 | CCAR1 |
| Melanoma | 0 | 0 | CCAR1 |
| Uterus | 2.06 | 1 | CCAR1 |
| Breast | 2.46 | 0 | CCAR1 |
| Kidney | 3.48 | 1 | CCAR1 |
| Uterus | 3.74 | 1 | CCAR1 |
| Melanoma | 4.89 | 1 | CCAR1 |
| Breast | 5.36 | 2 | CCAR1 |
| Lung and Bronchus | 6.33 | 1 | CCAR1 |
| Breast | 6.39 | 0 | CCAR1 |
| Lung | 8.05 | . | CCAR1 |
| Thyroid | 12.96 | 3 | CCAR1 |
| Breast | −17.08 | . | Negative |
| Uterus | −16.9 | . | Negative |
| Breast | −15.3 | . | Negative |
| Breast | −4.97 | 1 | Negative |
| Prostate | −4.53 | 2 | Negative |
| Colon | −4.09 | 3 | Negative |
| Breast | −2.66 | 2 | Negative |
| Thyroid | −2.2 | 3 | Negative |
| Uterus | −1.99 | . | Negative |
| NHL | −1.91 | 3 | Negative |
| NHL | −0.96 | . | Negative |
| Ovary | −0.94 | 4 | Negative |
| Breast | −0.721 | 0 | Negative |
| Ovary | −0.547 | 3 | Negative |
| Breast | −0.38 | 4 | Negative |
| Ovary | −0.281 | 3 | Negative |
| Melanoma | −0.235 | 0 | Negative |
| Esophagus | −0.172 | 3 | Negative |
| Prostate | −0.12 | 2 | Negative |
| Lung | 0.0136 | 4 | Negative |
| Breast | 0.076 | 1 | Negative |
| Breast | 0.136 | 1 | Negative |
| Nasopharyngeal | 0.167 | . | Negative |
| Ovary | 0.199 | 4 | Negative |
| Uterus | 0.251 | 3 | Negative |
| Lung | 0.257 | 2 | Negative |
| Bladder | 0.312 | 2 | Negative |
| Breast | 0.328 | 2 | Negative |
| NHL | 0.407 | 1 | Negative |
| Kidney | 0.443 | . | Negative |
| Breast | 0.449 | 1 | Negative |
| Colon | 0.563 | 2 | Negative |
| Ovary | 0.569 | 3 | Negative |
| Breast | 0.662 | 2 | Negative |
| Breast | 0.676 | 1 | Negative |
| Melanoma | 0.728 | 0 | Negative |
| Thyroid | 0.747 | . | Negative |
| Colon | 0.788 | 1 | Negative |
| Colon | 0.845 | 2 | Negative |
| Skin (NHL) | 0.9 | . | Negative |
| Hodgkin's Lymphoma | 0.958 | 2 | Negative |
| Breast | 1.33 | 0 | Negative |
| Hodgkin's Lymphoma | 1.46 | 2 | Negative |
| Colon | 1.49 | 1 | Negative |
| Esophagus | 1.579 | . | Negative |
| Colon | 1.61 | 4 | Negative |
| Uterus | 2.04 | 4 | Negative |
| Breast | 2.12 | 2 | Negative |
| Breast | 2.19 | 4 | Negative |
| Thyroid | 2.41 | . | Negative |
| Desmoid Tumor | 2.87 | 0 | Negative |
| NHL | 3.16 | 2 | Negative |
| Oropharynx/SCC | 3.19 | 1 | Negative |
| Breast | 4.2 | 0 | Negative |
| Multiple Myeloma | 6.79 | 1 | Negative |
| Breast | 7.24 | 0 | Negative |
| Ovary | 9.34 | 4 | Negative |
| Oropharynx/SCC | 9.53 | 4 | Negative |
| Lung | 10.06 | 2 | Negative |
| Uterus | 10.3 | 1 | Negative |
| Prostate | 11.2 | 2 | Negative |
| Breast | 16 | 2 | Negative |
| Breast | 18.65 | 1 | Negative |

Cancer type, timing, stage and autoantibody status of individual patients. NHL, non-Hodgkin's lymphoma; SCC, squamous cell carcinoma. “.” Indicates data not available.

A total of 10 anti-CCAR1-positive patients with cancer met these criteria, 9 of whom had staging data (Table 7). Analysis was restricted to cancers diagnosed between DM symptom onset and 10 years follow-up. Patients were included if there was information regarding stage at cancer diagnosis and were pooled from both the validation and discovery cohorts. Of these, 9 patients, 8 (89%) were diagnosed at low stage (0 or 1) and only 1 patient (11%) had a stage of 2 or greater. In contrast, patients with TIF1-γ autoantibodies alone had significantly fewer cancers at low stage, 14/33 (42%), p=0.02. While cancer types were largely similar in the anti-CCAR1-positive versus anti-CCAR1-negative autoantibody groups, the anti-CCAR1-negative group had 3 ovarian cancers, whereas the anti-CCAR1-positive group had none. In a sensitivity analysis excluding the cases of ovarian cancer, the enrichment of low stage cancers in the anti-CCAR1-positive group remained significant (p=0.05, Fischer exact test).

TABLE 7
Frequency of high versus low stage cancers
stratified by anti-CCAR1 antibody status.

| | TNM Stage 0 or 1 at | TNM Stage 2, 3, or 4 at |
|---|---|---|
| | Cancer Diagnosis | Cancer Diagnosis |
| Autoantibody Status | N (%) | N (%) |
| Anti-CCAR1-Positive | 8/9 (89%) | 1/9 (11%) |
| Anti-CCAR1-Negative | 14/33 (42%) | 19/33 (58%) |

The time interval between DM symptom onset and cancer appearance was examined (FIG. 3D). Patients positive for anti-CCAR1 antibodies were diagnosed with cancer significantly later compared to anti-CCAR1-negative patients (median time from DM onset 4.3 vs 0.85 years, respectively, p=0.006). This cannot be explained by differences in follow-up time, as this was similar in anti-CCAR1-positive and anti-CCAR1-negative cancer-free patients in both cohorts (validation cohort, median follow up 8 years in anti-CCAR1-negative patients, 10 years in anti-CCAR1-positive; discovery cohort, median follow up 9 years in anti-CCAR1-negative patients, 10 years in anti-CCAR1-positive).

For both the stage and time analyses, sensitivity analyses were performed to include cancers occurring 6 months prior to DM-symptom onset. Cancers occurring within 6 months of DM-symptom onset were considered to be contemporaneous in this sensitivity analysis. An additional 5 anti-CCAR1-negative and no anti-CCAR1-positive patients were included in “time zero”. Of these 5 anti-CCAR1-negative patients, 4 (80%) had a cancer stage of 2, 3, or 4. Including a comparison of stage (89% vs 39%, p=0.01 by Fischer Exact) showed similar results. Also, the time analysis in anti-CCAR1-positive vs negative patients, the median time of cancer diagnosis from DM onset was 4.3 vs 0.74 year, respectively p=0.002).

Additional Novel Antibodies and their Relationship with Cancer

Whether anti-CCAR1 antibodies were enriched in patients in whom a cancer never emerges, or emerges after a time delay was tested for the other autoantibody specificities identified within the anti-TIF1-γ-positive population (FIG. 4C). Of the 9 additional new specificities, 3 were found in the index case alone (anti-GATD1, anti-RCC1, and anti-KDM2A), while one was found only in the index case plus an additional patient (anti-NVL2). The remaining 5 were detected in multiple patients, with frequencies ranging from ˜2.5-21% of the anti-TIF1-γ-positive patients (anti-NACC1, anti-CIZ1, anti-IMMT, anti-TBL1XR1, and anti-SOX5).

Comparing the two cohorts, a similarity in the prevalence and rank-order of the 10 specificities was observed (FIG. 4C). In both cohorts, approximately half of the patients produced autoantibodies in addition to TIF1-γ; 30% produced one, and ˜20% produced two or more. Upon dichotomizing the 10 autoantibodies to zero (TIF1-γ only) versus any (TIF1-Y “plus”), large differences in cancer frequency were observed. In the validation cohort, there was a 4-fold higher frequency of cancer in the TIF1-γ only group compared to patients who produced any of the 10 autoantibodies: cancer emerged in 37% of patients with TIF1-γ only vs 9% TIF1-γ “plus” within 5 years, 34% vs 7% within 3 years, and 27% vs 4% within 1 year. In the discovery cohort, the cancer frequency was 2-fold higher: cancer emerged in 32% vs 19% within 5 years, 27% vs 13% within 3 years, and 20% vs 4% within 1 year).

The number of autoantibody specificities patients produced in relationship to cancer diagnosis was examined in the combined cohorts (Table 8). Patients with cancer from both cohorts were combined for analysis, and examined in both ±3 and ±1 time windows. For all DM-onset/cancer time intervals, a dose-response relationship was observed: as the number of autoantibody specificities patients produced increased, the frequency of cancer decreased. These trends were most notable for cancer within 3 and 1 year (Table 8; Fisher Exact p<0.001 for all trends).

TABLE 8
Frequency of cancer stratified by increasing number of autoantibodies.
Combined Discovery and Validation Cohorts

| # of | Cancer (±3) | | | Cancer (±1) | | |
|---|---|---|---|---|---|---|
| Autoantibodies | No (n = 203) | | Yes (n = 49) | No (n = 219) | | Yes (n = 33) |
| 0 | 82 | (70) | 36 (30) | 90 | (76) | 28 (24) |
| 1 | 68 | (85) | 12 (15) | 75 | (94) | 5 (6) |
| 2 | 36 | (97) | 1 (3) | 37 | (100) | 0 (0) |
| 3 | 14 | (100) | 0 (0) | 14 | (100) | 0 (0) |
| 4 | 2 | (100) | 0 (0) | 2 | (100) | 0 (0) |
| 5 | 1 | (100) | 0 (0) | 1 | (100) | 0 (0) |

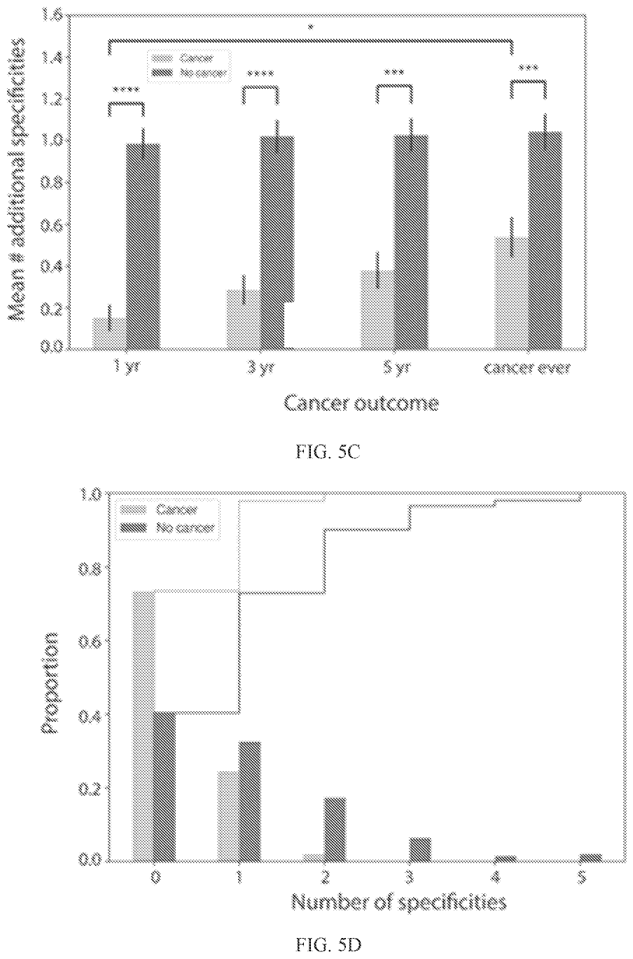

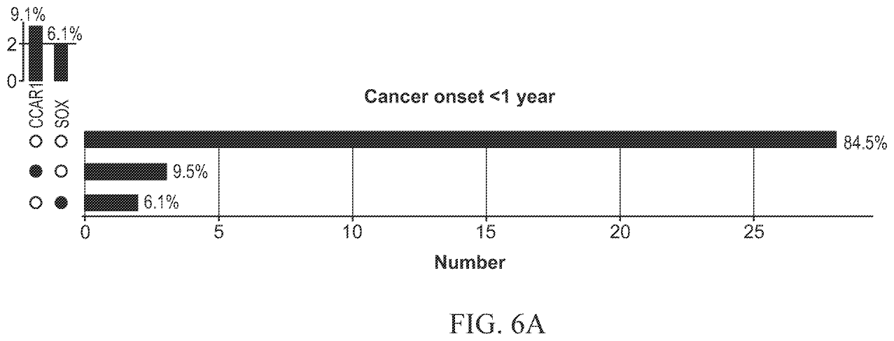

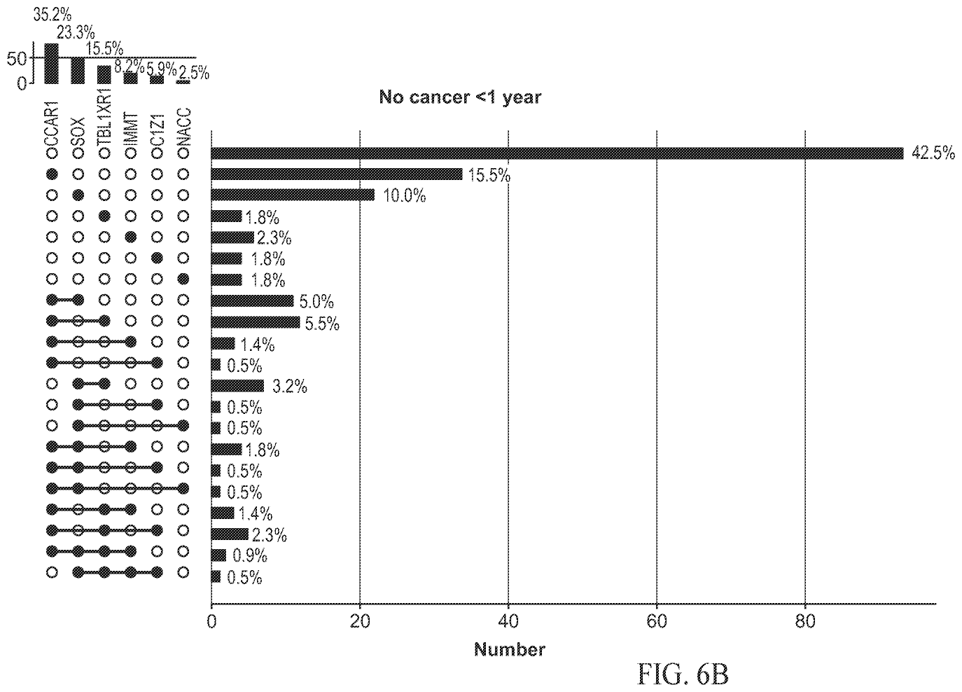

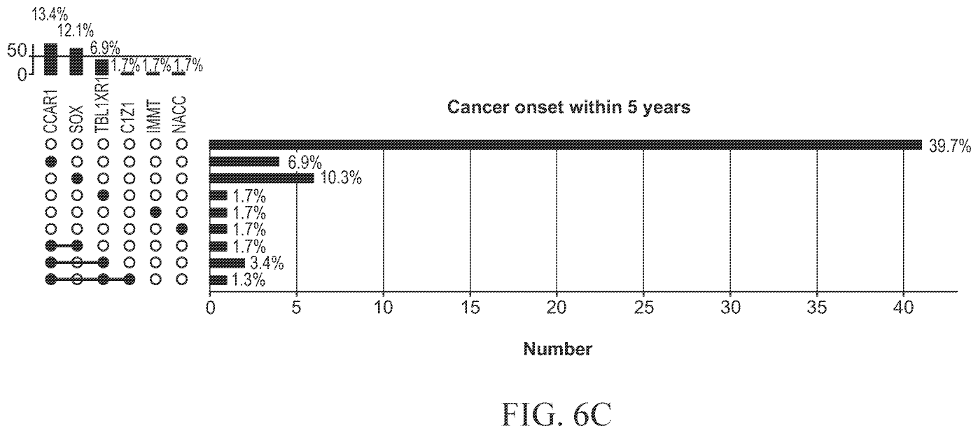

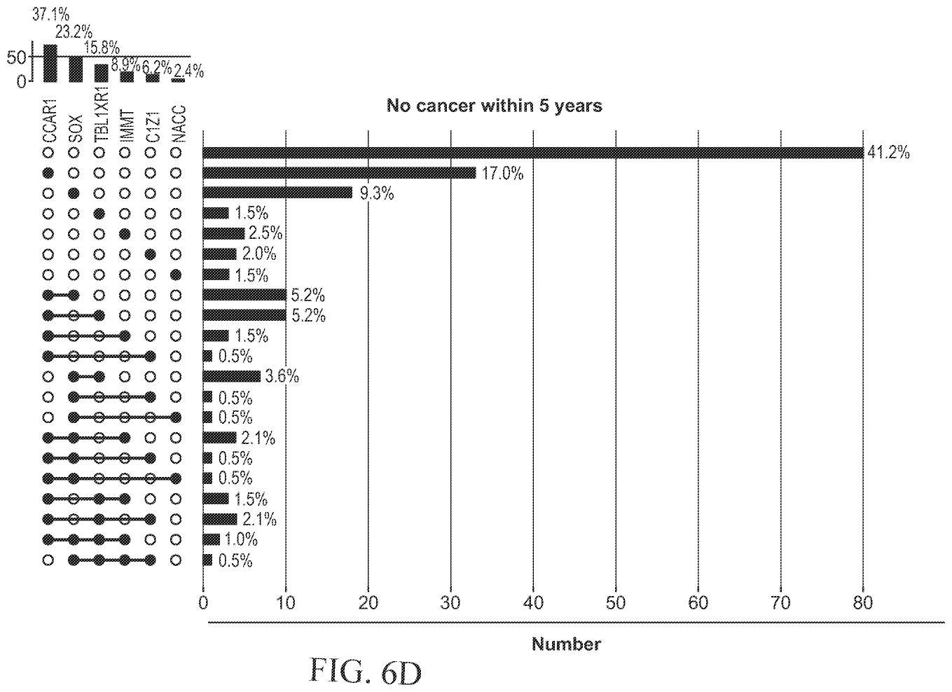

To further understand the relationship between combinations of autoantibodies and cancer emergence, the distribution of all such combinations in patients with versus without cancer at different intervals around DM onset was visualized using UpSet, a visualization tool for the quantitative analysis of overlapping subsets (FIG. 5A-5D and FIG. 6A-6D); see, for example, Lex A et al. IEEE Trans Vis Comput Graph. 2014; 20(12): 1983-92). Several features were evident:
(i) In patients with cancer ±3 year, 74% had TIF1-γ antibodies alone. The remaining 26% had either anti-CCAR1 or anti-SOX5 in isolation, and only 1 of these patients (2% of the group with cancer) had them in combination with another antibody (FIG. 5A). In contrast, only 42% of those without cancer at 3 years had TIF1γ antibodies alone (FIG. 5B). Some patients in this group had single additional antibodies from the group of 10 novel autoantibodies, most frequently against CCAR1 (16%) or SOX5 (9%). Particularly striking were the 14 combinations of multiple autoantibodies (FIG. 5B). These were present in 25.4% of patients and always involved combinations including either CCAR1 (11.4%) or SOX5 (4.8%), or both (9.2%).
(ii) While anti-CCAR1 antibodies in isolation were enriched in patients without cancer, anti-SOX5 autoantibodies in isolation did not have a similar association.
(iii) The mean number of additional autoantibody specificities in DM patients with cancer was 0.15 in patients with cancer ±1 year, rising to 0.38 in patients with cancer ±5 years. In contrast, the mean number of additional specificities for patients without cancer was 1 at all of the time points. The difference was statistically significant at all of the time points (FIG. 5C).
(iv) When anti-CCAR1 antibodies were present, they occurred alone in 46% (37/80) of patients, and in combination in 54% (43/80). Similarly, isolated anti-SOX5 antibodies occurred in 45% (24/53) of anti-SOX5-positive patients, and in combination in 55% (29/53). Anti-TBL1XR1 antibodies were present in isolation in 11.7% (4/34) of patients, and were found in combinations with other specificities in 88.3% (30/34) (distributions between solo and combination were different for anti-TBL1XR1 and anti-CCAR1 (P<0.0005), as well as anti-TBL1XR1 and SOX5 (P<0.001)). Anti-TBL1XR1 autoantibodies therefore appear to arise in the setting of immune responses against CCAR1, SOX5, or both.

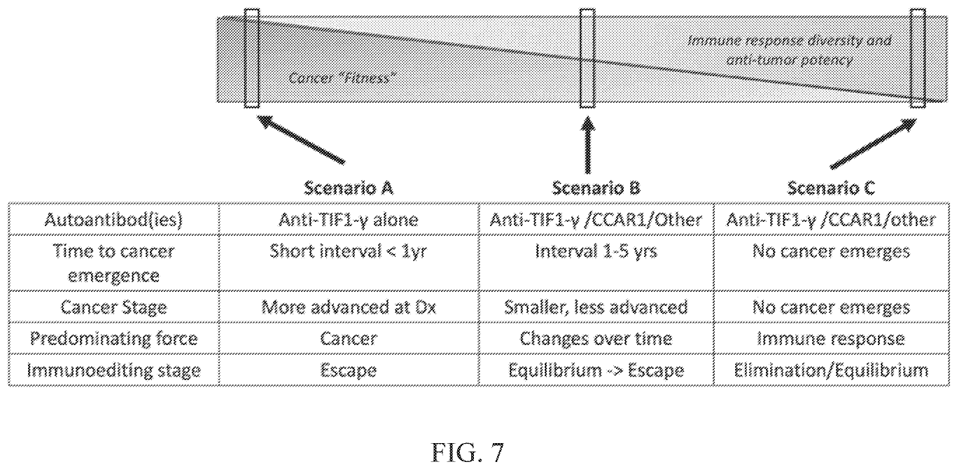

An exemplary cancer model of immunoediting depicted as a spectrum of decreasing cancer fitness and its inverse relationship with the anti-tumor immune response is in FIG. 7. All scenarios represent DM in association with anti-TIF1-γ antibodies and incipient cancer. Scenario A represents a state of high cancer fitness with a paucity of additional immune responses beyond anti-TIF1-γ. This part of the spectrum is associated with rapid (around time of DM onset) and aggressive (e.g. advanced stage) cancer emergence (“immune escape”). Scenario B represents a balance between cancer and immune response (equilibrium), and is characterized by a broader immune response (e.g. anti-CCAR1). In this scenario, cancer eventually manifests (a transition from equilibrium to immune escape), but is less aggressive (e.g. earlier stage) and emerges after a time delay following DM onset. Scenario C is also characterized by a broad (e.g. anti-CCAR1) and effective immune response, but is one in which the anti-tumor response ultimately deletes (elimination) or maintains the cancer in a subclinical state (equilibrium).

### Example 2: ELISA Assay to Detect Antibodies Against CCAR1 in Human Sera

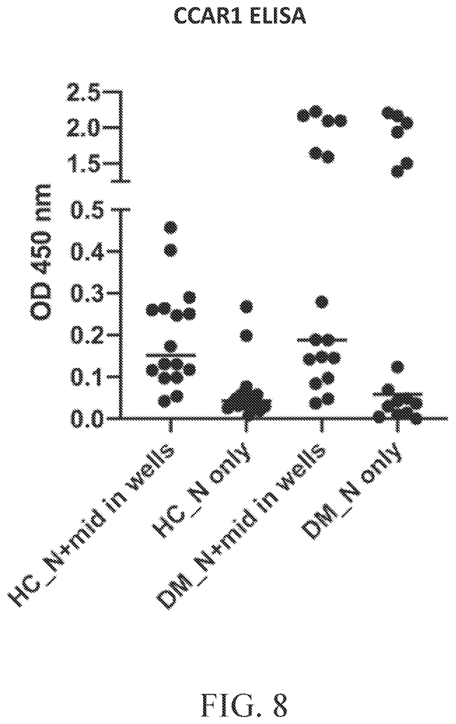

A study was performed to determine which piece of recombinant human CCAR1 protein performed better in an ELISA assay for detection of anti-CCAR1 antibodies. The following 2 pieces of recombinant human CCAR1 protein were tested for detection by ELISA: amino acids 1-234 (“N-terminal piece”) and amino acids 188-645 (“mid region”). ELISA wells were coated with either the N-terminal CCAR1 piece only (50 nanograms/well), or a mix of the N-term and mid-region CCAR1 pieces (50 nanograms of each/well). Sera from 16 healthy controls and 16 patients with DM were tested. Cutoffs for positive antibody status were defined as the mean OD+3SD of the healthy controls. The cutoff of wells coated with N+mid CCAR1 was 0.558 OD units. The cutoff for wells coated with N-term CCAR1 only was 0.273 OD units. Results from assays using both coatings were consistent (FIG. 8). The same 6 DM sera were positive using both types of well coatings.

In another study the wells in 96-well ELISA plates were coated overnight with 50 ng/well of the N-terminal piece of human CCAR1 (amino acids 1-234). The wells were washed three times with PBS/Tween (PBST), and blocked at room temperature with 5% BSA/PBST (2 hours), followed by three PBST washes. Sera were diluted 1:400 in 1% BSA/PBST, added to the wells, and incubated at room temperature for 1 hour. After three PBST washes, HRP-conjugated anti-human IgG secondary antibody (diluted 1:10,000 in 1% BSA/PBST) was added to the wells and incubated for 1 hour at room temperature. Wells were then washed three times with PBST, followed by two washes with PBS only. Color was developed by incubating the plates with the SureBlue reagent for 20 minutes at room temperature, then hydrochloric acid was added. ODs were read in a plate reader at 450 nanometers. A positive reference serum was included on each ELISA plate as a calibrator.

The ELISA method was used to assay a set of 32 healthy control sera. Cutoffs for positive anti-CCAR1 antibody status were calculated (OD-0.307). 107 DM sera samples were tested using the above ELISA assay, and also tested using the IP assay. Input for the IP assay is 35S-methionine labeled full-length human CCAR1 generated by in vitro transcription and translation. Antibody statuses measured using the IP and ELISA methods were consistent for all 107 sera samples (Table 9).

TABLE 9
Comparing IP with IVTT Full Length CCAR1 and
ELISA with N-terminal piece coating wells.

| serum | IVTTIP | ELISA (OD) |
|---|---|---|
| 4002 | neg | neg |
| 5008 | neg | neg |
| 5013 | neg | neg |
| 5020 | neg | neg |
| 6027 | neg | neg |
| 6050 | pos | 2.21 |
| 6065 | neg | neg |
| 7005 | pos | 2.16 |
| 7018 | pos | 1.5 |
| 7020 | neg | neg |
| 7080 | neg | neg |
| 7095 | neg | neg |
| 7100 | neg | neg |
| 7104 | neg | neg |
| 8012 | neg | neg |
| 8049 | neg | neg |
| 8093 | neg | neg |
| 8101 | neg | neg |
| 8122 | faint pos | 0.81 |
| 8147 | pos | 1.61 |
| 8152 | neg | neg |
| 8156 | neg | neg |
| 8169 | neg | neg |
| 8193 | neg | neg |
| 8200 | neg | neg |
| 8226 | neg | neg |
| 9017 | neg | neg |
| 9062 | neg | neg |
| 9067 | neg | neg |
| 9070 | neg | neg |
| 9073 | neg | neg |
| 9078 | neg | neg |
| 9080 | neg | neg |
| 9114 | neg | neg |
| 9116 | neg | neg |
| 9152 | neg | neg |
| 9160 | neg | neg |
| 9164 | neg | neg |
| 9189 | neg | neg |
| 9199 | neg | neg |
| 9201 | neg | neg |
| 9209 | neg | neg |
| 10102 | pos | 1.313 |
| 10103 | neg | neg |
| 10138 | neg | neg |
| 10175 | neg | neg |
| 10187 | pos | 1.401 |
| 11008 | neg | neg |
| 11019 | neg | neg |
| 11056 | pos | 1.131 |
| 11077 | pos | 1.277 |
| 11087 | neg | neg |
| 11096 | neg | neg |
| 11112 | neg | neg |
| 11136 | neg | neg |
| 12010 | neg | neg |
| 12013 | neg | neg |
| 12038 | neg | neg |
| 12050 | neg | neg |
| 12067 | neg | neg |
| 12081 | pos | 2.655 |
| 12088 | pos | 1.582 |
| 12097 | neg | neg |
| 12146 | pos | 1.113 |
| 12161 | neg | neg |
| 12166 | neg | neg |
| 12171 | neg | neg |
| 12181 | pos | 2.816 |
| 12187 | pos | 1.366 |
| 12203 | neg | neg |
| 13020 | neg | neg |
| 13034 | neg | neg |
| 13108 | pos | 2.348 |
| 13142 | neg | neg |
| 13195 | neg | neg |
| 13255 | neg | neg |
| 14021 | neg | neg |
| 14028 | pos | 2.897 |
| 14030 | pos | 1.169 |
| 14044 | neg | neg |
| 14049 | neg | not tested |
| 14053 | neg | neg |
| 14061 | neg | neg |
| 14064 | neg | neg |
| 14082 | pos | 0.645 |
| 14113 | neg | neg |
| 14115 | neg | neg |
| 14173 | neg | neg |
| 14202 | neg | neg |
| 14207 | neg | neg |
| 14221 | neg | neg |
| 14240 | neg | neg |
| 15014 | pos | 1.812 |
| 15040 | neg | neg |
| 15046 | pos | 1.3 |
| 15074 | pos | 1.867 |
| 15101 | pos | 1.467 |
| 15103 | neg | neg |
| 16017 | neg | neg |
| 16062 | pos | 2.077 |
| 16063 | neg | neg |
| 16137 | neg | neg |
| 16152 | pos | 2.503 |
| 16165 | neg | neg |
| 17056 | neg | neg |
| 17153 | neg | neg |
| 18020 | neg | neg |
| 18083 | faint pos | 0.715 |

### Example 3: Treating Cancer

A blood sample (e.g., serum or plasma) is obtained from a human having DM. The sample is examined for the presence of anti-TIF1-γ antibodies and the presence of anti-CCAR1 antibodies. In some cases, an ELISA assay is performed to detect the presence of anti-TIF1-γ antibodies and the presence of anti-CCAR1 antibodies. If the presence of anti-TIF1-γ antibodies and the absence of anti-CCAR1 antibodies are detected in the sample, then the human is administered one or more inhibitors of a CCAR1 polypeptide (e.g., anti-CCAR1 antibodies). The administered inhibitor(s) can delay or prevent the development of a cancer within the human.

### Example 4: Treating Cancer

A blood sample (e.g., serum or plasma) is obtained from a human having DM. The sample is examined for the presence of anti-TIF1-γ antibodies and the presence of anti-CCAR1 antibodies. In some cases, an ELISA assay is performed to detect the presence of anti-TIF1-γ antibodies and the presence of anti-CCAR1 antibodies. If the presence of anti-TIF1-γ antibodies and the absence of anti-CCAR1 antibodies are detected in the sample, then the human is administered one or more inhibitors of a CCAR1 polypeptide (e.g., anti-CCAR1 antibodies). The administered inhibitor(s) can reduce or eliminate the number of cancer cells present within the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-15. (canceled)

16. A method for treating a mammal having cancer, said method comprising:
detecting a presence of an anti-TIF1-γ antibody and an absence of a decreased cancer risk (DCR) antibody in a sample obtained from said mammal; and
administering an inhibitor of a DCR polypeptide to said mammal.

17. A method for treating a mammal having cancer, the method comprising administering an inhibitor of a CCAR1 polypeptide to a mammal identified as having a presence of an anti-TIF1-γ antibody and an absence of a DCR antibody.

18. The method of claim 16, wherein said mammal is a human.

19. The method of claim 16, wherein said mammal has an autoimmune disease.

20. The method of claim 19, wherein said autoimmune disease is DM.

21. The method of claim 16, wherein the DCR antibody comprises each of an anti-CCAR1 antibody, an anti-GATD1 antibody, an anti-TBL1XR1 antibody, an anti-KDM2A antibody, an anti-IMMT antibody, an anti-SOX5 antibody, an anti-CIZ1 antibody, an anti-NVL2 antibody, and an anti-NACC1 antibody.

22. The method of claim 21, wherein the DCR antibody is an anti-CCAR1 antibody.

23. The method of claim 16, wherein said inhibitor of said CCAR1 polypeptide is an anti-CCAR1 antibody.

24. The method of claim 16, wherein said cancer is selected from the group consisting of prostate cancer, breast cancer, leukemia cancer, lung cancer, melanoma, uterine cancer, kidney cancer, thyroid cancer, ovarian cancer, and colon cancer.

25-29. (canceled)

30. A method for detecting a presence or absence of an anti-CCAR1 antibody in a sample, said method comprising:
contacting said sample to a CCAR1 polypeptide fragment consisting essentially of or consisting of the amino acid sequence set forth in SEQ ID NO:1 or SEQ ID NO:2 under conditions that allow a complex to form between said CCAR1 polypeptide fragment and said anti-CCAR1 antibody; and
detecting said complex wherein said complex is indicative of the presence of said anti-CCAR1 antibody.

31. The method of claim 30, wherein said CCAR1 polypeptide fragment is immobilized on an enzyme-linked immunosorbent assay (ELISA) plate.

32. An ELISA plate, wherein a CCAR1 polypeptide fragment consisting essentially of or consisting of the amino acid sequence set forth in SEQ ID NO:1 or SEQ ID NO:2 is immobilized on said ELISA plate.
